(19) United States
(12) Patent Application Publication
(10) Pub. No.: US 2015/0352018 A1
(43) Pub. Date: Dec. 10, 2015

(54) ENCODED PARTICLES COMPOSITIONS

(71) Applicant: HLAVIN INDUSTRIES LTD, Zur Yigal (IL)

(72) Inventors: Karny HLAVIN, Herzeliya (IL); Eduard POLITKIN, Holon (IL)

(73) Assignee: HLAVIN INDUSTRIES LTD, Zur Yigal (IL)

(21) Appl. No.: 14/758,573

(22) PCT Filed: Dec. 31, 2013
(86) PCT No.: PCT/IL2013/051101

§ 371 Date: Jun. 30, 2015

Related U.S. Application Data
(60) Provisional application No. 61/747,401, filed on Dec. 31, 2012.

(51) Int. Cl. A61K 8/19; A61K 33/10; A61N 2/00; A61K 9/00; A61Q 19/00; A61N 2/02; A61K 31/7016; A61K 8/60

(52) U.S. Cl. CPC A61K 8/19; A61K 31/7016; A61K 33/10; A61K 8/60; A61K 9/0014; A61Q 19/00; A61Q 19/007; A61N 2/02; A61N 2/002; A61K 2800/83; A61K 2800/70

(57) ABSTRACT

The present invention provides a composition comprising at least one type of irradiated crystals and an aqueous preparation. The aforementioned irradiated crystals are exposed to a changing electromagnetic field. The present invention further relates to methods and processes for preparing and using the compositions.

### CROSS REFERENCE

This application is a national phase of PCT Application Number PCT/IL2013/051101, filed on Dec. 31, 2013, which claims priority from provisional application No. 61/747,401, filed on Dec. 31, 2012. All of these applications are hereby incorporated by reference in their entirety.

### FIELD OF THE INVENTION

This invention is directed towards compositions and the processes for preparing and using them. More specifically, the present invention relates to composition comprising irradiated crystals such as smart crystals or pearls for skin recovery and antiaging treatment.

### BACKGROUND OF THE INVENTION

Since time immemorial there have been many attempts to provide topical preparations for cosmetic solutions to aging and skin disorders.

Patent application WO2010/013191 provides a system combining creams and lotions to be applied to the skin, which are activated by irradiating the skin. Such systems are inconvenient and require special equipment for treatment.

There is therefore a long felt and unmet need to provide improved efficacious compositions, processes and methods for antiaging which can be applied by consumers conveniently.

### SUMMARY OF THE INVENTION

It is one object of the present invention to disclose a topical composition for skin improvement, said composition characterized by at least one type of irradiated crystals and an aqueous preparation, the crystal irradiated according to at least one frequencies scheme selected from the group consisting of a range of between about 0.1 and 1000 Hz, about 0.2 and 200 Hz, a range of between about 1 and 150 Hz and any combination thereof; said improvement is selected from the group consisting of increasing skin biologic elasticity, decreasing skin viscoelastic, increasing skin hydration, decreasing of skin wrinkles and skin fine lines, skin regeneration and any combination thereof.

It is one object of the present invention to disclose a topical composition for providing a skin improvement, the composition characterized by at least one type of irradiated crystals and an aqueous preparation, the crystal irradiated according to a predetermined intensities range selected from the group consisting of a range of 1 and 1000 μT, a range of 10 and 500 μT and any combination thereof; the improvement is selected from the group consisting of increasing skin biologic elasticity, decreasing skin viscoelastic, increasing skin hydration, decreasing of skin wrinkles and skin fine lines, skin regeneration and any combination thereof.

It is a further object of the present invention to disclose the composition as defined in any of the above, wherein the frequencies scheme includes a first sequence of the frequencies increasing linearly and continuously from a first frequency to a second, greater, frequency, and a second sequence of the frequencies decreasing linearly and continuously from the second frequency to the first frequency.

It is a further object of the present invention to disclose the composition as defined in any of the above, wherein the composition when applied topically associates with an increase of more than 9% in skin biologic elasticity.

It is a further object of the present invention to disclose the composition as defined in any of the above, wherein the composition when applied topically correlates with a decrease of at least 8% in skin viscoelastic portion of the deformation process and an increase of the elastic properties, respectively. It is a further object of the present invention to disclose the composition as defined in any of the above, wherein the composition when applied topically increase of more than 15% of skin hydration.

It is a further object of the present invention to disclose the composition as defined in any of the above, wherein the composition when applied topically provides a decrease of at least 10% of skin profilometry, thereby improvement of wrinkle and fine line appearance of the skin.

It is a further object of the present invention to disclose the composition as defined in any of the above, wherein the crystal is made of sucrose, silicon, pearl smart pearl, quartz, smart patches, smart particles, encoded gems, encoded stones, encoded food supplements or any other inert material and a combination thereof.

It is a further object of the present invention to disclose the composition as defined in any of the above, wherein the aqueous preparation is in the form of a lotion, mixture, compound, formulation, gel, cream, serum, liquid infusion, spray, suspension, emulsion, emollient preparation, emollient moisturiser and any combination thereof.

It is a further object of the present invention to disclose the composition as defined in any of the above, wherein the skin improvements further includes parameters and/or disorders selected from the group consisting of trophism of the integumentary system, regeneration of skin cells, skin irritation, skin infection, skin inflammation, the effectiveness of the skin as a barrier, skin aging, skin pigmentation, allergy, dermatitis, allergic dermatitis, skin degeneration or atrophy and any combination thereof.

It is another object of the present invention to disclose a method of providing a skin improvement selected from the group consisting of increasing skin biologic elasticity, decreasing skin viscoelastic, increasing skin hydration, decreasing of skin wrinkles and skin fine lines, skin regeneration and any combination thereof; the method comprising steps of: applying a composition comprising at least one type of irradiated crystals and an aqueous preparation; the irradiated crystals are exposed to a a frequencies scheme selected from the group consisting of a range of between about 0.1 and 1000 Hz, about 0.2 and 200 Hz, a range of between about 1 and 150 Hz and any combination thereof.

It is another object of the present invention to disclose a method of providing a skin improvement selected from the group consisting of increasing skin biologic elasticity, decreasing skin viscoelastic, increasing skin hydration, decreasing of skin wrinkles and skin fine lines, skin regeneration and any combination thereof; the method comprising steps of: applying a topical composition comprising at least one type of irradiated crystals and an aqueous preparation; the irradiated crystals are exposed to a predetermined intensities range selected from the group consisting of a range of 1 and 1000 μT, a range of 10 and 500 μT and any combination thereof.

It is a further object of the present invention to disclose the method as defined in any of the above, wherein the method additionally comprising step of providing the irradiated crystals exposed to frequencies scheme including a first sequence of the frequencies increasing linearly and continuously from a first frequency to a second, greater, frequency, and a second sequence of the frequencies decreasing linearly and continuously from the second frequency to the first frequency.

It is another object of the present invention to disclose a composition comprising at least one type of irradiated crystals and an aqueous preparation wherein the irradiated crystals are exposed to a changing electromagnetic field.

It is a further object of the present invention to disclose the composition as defined in any of the above, wherein the changing electromagnetic field is applied according to a predetermined protocol.

It is a further object of the present invention to disclose the composition as defined in any of the above, wherein the changing electromagnetic field oscillates at a frequency in the range of between about 0.1 and 1000 Hz.

It is a further object of the present invention to disclose the composition as defined in any of the above, wherein the changing electromagnetic field oscillates at a frequency in the range of between about 0.2 and 200 Hz.

It is a further object of the present invention to disclose the composition as defined in any of the above, wherein the changing electromagnetic field oscillates at a frequency in the range of between about 1 and 150 Hz.

It is a further object of the present invention to disclose the composition as defined in any of the above, wherein the changing electromagnetic field is applied in increased frequencies.

It is a further object of the present invention to disclose the composition as defined in any of the above, wherein the changing electromagnetic field is applied in a series of predetermined intensities in the range of 1 and 1000 μT.

It is a further object of the present invention to disclose the composition as defined in any of the above, wherein the changing electromagnetic field is applied in a series of predetermined intensities in the range of 10 and 500 μT, preferably 5 to 200 μT.

It is a further object of the present invention to disclose the composition as defined in any of the above, wherein the changing electromagnetic field is applied in a continuous waveform.

It is a further object of the present invention to disclose the composition as defined in any of the above, wherein the changing electromagnetic field is applied in a pulsed waveform.

It is a further object of the present invention to disclose the composition as defined in any of the above, wherein the crystal is made of sucrose, silicon, pearl, smart pearl, quartz, smart patches, smart particles, encoded gems, encoded stones, encoded food supplements or any other inert material and a combination thereof.

It is a further object of the present invention to disclose the composition as defined in any of the above, wherein the aqueous preparation is in the form of a lotion, mixture, compound, formulation, gel, cream, serum, liquid infusion, spray, suspension, emulsion, emollient preparation, emollient moisturiser and any combination thereof.

It is a further object of the present invention to disclose the composition as defined in any of the above, wherein the composition is adapted to improve or treat at least one medical parameter or condition in a mammal, preferably in a human.

It is a further object of the present invention to disclose the composition as defined in any of the above, wherein the composition is adapted to prevent at least one medical condition in a mammal, preferably in a human.

It is a further object of the present invention to disclose the composition as defined in any of the above, wherein the composition is adapted to stimulate the regeneration and/or the recovery of an organ, tissue or a system or a portion thereof in a mammal, preferably in a human.

It is a further object of the present invention to disclose the composition as defined in any of the above, wherein the medical parameter or condition is selected from the group consisting of skin related parameters and/or disorders, recovery of an organ, tissue or system or a portion thereof, brain related parameters and any combination thereof.

It is a further object of the present invention to disclose the composition as defined in any of the above, wherein the skin related parameters and/or disorders are selected from the group consisting of trophism of the integumentary system, regeneration of skin cells, skin irritation, skin infection, skin inflammation, the effectiveness of the skin as a barrier, skin aging, skin pigmentation, allergy, dermatitis, allergic dermatitis, skin degeneration or atrophy and any combination thereof.

It is a further object of the present invention to disclose the composition as defined in any of the above, wherein the organ, tissue or system is selected from the group consisting of the lymph system, the endocrine system, the liver, skin, the integumentary system and any portion or combination thereof.

It is a further object of the present invention to disclose the composition as defined in any of the above, wherein the brain related parameters are selected from the group consisting of regulation and/or controlling of the muscle tonus center and/or motor control center of the cerebellum, the reflex centers, regulation or controlling of the posterior pituitary (Neurohypophysis) of the pituitary gland or hypophysis, regulation of the skin center in the brain and any combination thereof.

It is a further object of the present invention to disclose the composition as defined in any of the above, wherein the composition is applied topically to the integuments and covering or support tissues of mammals, in human or veterinary fields.

It is a further object of the present invention to disclose a process for the preparation of a composition for prevention, treatment, stimulation and/or recovery of at least a portion of a mammalian tissue or system, preferably the skin system, comprising the steps of: (a) providing an aqueous preparation; (b) providing at least one type of crystal; (c) exposing the at least one type of crystal to a changing electromagnetic field so as to obtain at least one type of an irradiated crystals; (d) contacting the at least one type of irradiated crystals with the aqueous preparation thereby producing the composition for prevention, treating, and/or stimulating the recovery of the at least a portion of a mammalian tissue or system, preferably the skin system.

It is a further object of the present invention to disclose the process as defined in any of the above, comprising an additional step of exposing the at least one type of crystal to a changing electromagnetic field according to a predetermined protocol.

It is a further object of the present invention to disclose the process as defined in any of the above, comprising an additional step of exposing the at least one type of crystal to a changing electromagnetic field oscillating at a frequency in the range of between about 0.1 and 1000 Hz.

It is a further object of the present invention to disclose the process as defined in any of the above, comprising an additional step of exposing the at least one type of crystal to a changing electromagnetic field oscillating at a frequency in the range of between about 0.2 and 200 Hz.

It is a further object of the present invention to disclose the process as defined in any of the above, comprising an additional step of exposing the at least one type of crystal to a changing electromagnetic field oscillating at a frequency in the range of between about 1 and 150 Hz.

It is a further object of the present invention to disclose the process as defined in any of the above, comprising an additional step of exposing the at least one type of crystal to a changing electromagnetic field applied in increased frequencies.

It is a further object of the present invention to disclose the process as defined in any of the above, wherein the step of exposing the at least one type of crystal to a changing electromagnetic field is applied in a series of predetermined intensities in the range of 1 and 1000 μT.

It is a further object of the present invention to disclose the process as defined in any of the above, wherein the step of exposing the at least one type of crystal to a changing electromagnetic field is applied in a series of predetermined intensities in the range of 10 and 500 μT, preferably 5 to 200 μT.

It is a further object of the present invention to disclose the process as defined in any of the above, wherein the step of exposing the at least one type of crystal to a changing electromagnetic field is applied in a continuous waveform.

It is a further object of the present invention to disclose the composition as defined in any of the above, wherein the step of exposing the at least one type of crystal to a changing electromagnetic field is applied in a pulsed waveform.

It is a further object of the present invention to disclose the composition as defined in any of the above, comprising an additional step of providing at least one type of crystal made of sucrose, silicon, pearl, smart pearl, quartz, smart patches, smart particles, encoded gems, encoded stones, encoded food supplements or any other inert material and a combination thereof.

It is a further object of the present invention to disclose the composition as defined in any of the above, comprising an additional step of providing an aqueous preparation in the form of a lotion, mixture, compound, formulation, gel, cream, liquid infusion, spray, suspension, emulsion, emollient preparation, emollient moisturiser and any combination thereof.

It is a further object of the present invention to disclose the composition as defined in any of the above, wherein the process complies with GMP reproducibility standards.

It is a further object of the present invention to disclose the composition as defined in any of the above, wherein the final composition product meets the standard GMP requirements.

It is a further object of the present invention to disclose a method for prevention, treatment, stimulation and/or recovery of at least a portion of a mammalian tissue or system, preferably the skin system, wherein the method comprising the steps of: (a) providing a composition comprising at least one type of irradiated crystals and an aqueous preparation wherein the irradiated crystals are exposed to a changing electromagnetic field and, (b) applying the composition topically to at least a portion of the integuments, covering or support tissues of mammals so as to prevent, treat, and/or stimulate the recovery of the at least a portion of a mammalian tissue or system, preferably the skin system.

It is a further object of the present invention to disclose the method as defined in any of the above, comprising an additional step of applying the changing electromagnetic field according to a predetermined protocol.

It is a further object of the present invention to disclose the method as defined in any of the above, comprising an additional step of exposing the at least one type of crystal to a changing electromagnetic field oscillating at a frequency in the range of between about 0.1 and 1000 Hz.

It is a further object of the present invention to disclose the method as defined in any of the above, comprising an additional step of exposing the at least one type of crystal to a changing electromagnetic field oscillating at a frequency in the range of between about 0.2 and 200 Hz.

It is a further object of the present invention to disclose the method as defined in any of the above, comprising an additional step of exposing the at least one type of crystal to a changing electromagnetic field oscillating at a frequency in the range of between about 1 and 150 Hz.

It is a further object of the present invention to disclose the method as defined in any of the above, comprising an additional step of applying the changing electromagnetic field in increased frequencies.

It is a further object of the present invention to disclose the method as defined in any of the above, comprising an additional step of applying the changing electromagnetic field in a series of predetermined intensities in the range of 1 and 1000 μT.

It is a further object of the present invention to disclose the method as defined in any of the above, comprising an additional step of applying the changing electromagnetic field in a series of predetermined intensities in the range of 10 and 500 μT, preferably 5 to 200 μT.

It is a further object of the present invention to disclose the method as defined in any of the above, comprising an additional step of applying the changing electromagnetic field in a continuous waveform.

It is a further object of the present invention to disclose the method as defined in any of the above, comprising an additional step of applying the changing electromagnetic field in a pulsed waveform.

It is a further object of the present invention to disclose the method as defined in any of the above, comprising an additional step of providing the crystal made of sucrose, silicon, pearl, smart pearl, quartz, smart patches, smart particles, encoded gems, encoded stones, encoded food supplements or any other inert material and a combination thereof.

It is a further object of the present invention to disclose the method as defined in any of the above, comprising an additional step of preparing the aqueous preparation is in the form of a lotion, mixture, compound, formulation, gel, cream, liquid infusion, spray, suspension, emulsion, emollient preparation, emollient moisturizer and any combination thereof.

It is a further object of the present invention to disclose the method as defined in any of the above, comprising an additional step of improving or treating at least one medical parameter or condition in a mammal, preferably in a human.

It is a further object of the present invention to disclose the method as defined in any of the above, comprising an additional step of preventing at least one medical condition in a mammal, preferably in a human.

It is a further object of the present invention to disclose the method as defined in any of the above, comprising an additional step of stimulating the regeneration and/or the recovery of an organ, tissue or a system or a portion thereof in a mammal, preferably in a human.

It is a further object of the present invention to disclose the method as defined in any of the above, comprising an additional step of selecting the medical parameter or condition from the group consisting of skin related parameters and/or disorders, recovery of an organ, tissue or system or a portion thereof, brain related parameters and any combination thereof.

It is a further object of the present invention to disclose the method as defined in any of the above, comprising an additional step of selecting the skin related parameters and/or disorders from the group consisting of trophism of the integumentary system, regeneration of skin cells, skin irritation, skin infection, skin inflammation, the effectiveness of the skin as a barrier, skin aging, skin pigmentation, allergy, dermatitis, allergic dermatitis, skin degeneration or atrophy and any combination thereof.

It is a further object of the present invention to disclose the method as defined in any of the above, comprising an additional step of selecting the organ, tissue or system from the group consisting of the lymph system, the endocrine system, the liver, skin, the integumentary system and any portion or combination thereof.

It is a further object of the present invention to disclose the method as defined in any of the above, comprising an additional step of selecting the brain related parameters from the group consisting of regulation and/or controlling of the muscle tonus center and/or motor control center of the cerebellum, the reflex centers, regulation or controlling of the posterior pituitary (Neurohypophysis) of the pituitary gland or hypophysis, regulation of the skin center in the brain and any combination thereof.

It is a further object of the present invention to disclose the method as defined in any of the above, comprising an additional step of applying the composition topically to the integuments and covering or support tissues of mammals, in human or veterinary fields.

It is a further object of the present invention to disclose an irradiated crystal activated by exposure to a changing electromagnetic field, for topical use.

It is a further object of the present invention to disclose an irradiated crystal activated by exposure to a changing electromagnetic field, wherein the irradiated crystal is useful in cosmetic preparations.

It is a further object of the present invention to disclose the irradiated crystal as defined in any of the above, wherein the irradiated crystal is useful in aqueous cosmetic preparations.

It is a further object of the present invention to disclose the irradiated crystal as defined in any of the above, wherein the changing electromagnetic field is applied according to a predetermined protocol.

It is a further object of the present invention to disclose the irradiated crystal as defined in any of the above, wherein the changing electromagnetic field oscillates at a frequency in the range of between about 0.1 and 1000 Hz.

It is a further object of the present invention to disclose the irradiated crystal as defined in any of the above, wherein the changing electromagnetic field oscillates at a frequency in the range of between about 0.2 and 200 Hz.

It is a further object of the present invention to disclose the irradiated crystal as defined in any of the above, wherein the changing electromagnetic field oscillates at a frequency in the range of between about 1 and 150 Hz.

It is a further object of the present invention to disclose the irradiated crystal as defined in any of the above, wherein the changing electromagnetic field is applied in increased frequencies.

It is a further object of the present invention to disclose the irradiated crystal as defined in any of the above, wherein the changing electromagnetic field is applied in a series of predetermined intensities in the range of 1 and 1000 μT.

It is a further object of the present invention to disclose the irradiated crystal as defined in any of the above, wherein the changing electromagnetic field is applied in a series of predetermined intensities in the range of 10 and 500 μT, preferably 5 to 200 μT.

It is a further object of the present invention to disclose the irradiated crystal as defined in any of the above, wherein the changing electromagnetic field is applied in a continuous waveform.

It is a further object of the present invention to disclose the irradiated crystal as defined in any of the above, wherein the changing electromagnetic field is applied in a pulsed waveform.

It is a further object of the present invention to disclose the irradiated crystal as defined in any of the above, wherein the crystal is made of sucrose, silicon, pearl, smart pearl, quartz, smart patches, smart particles, encoded gems, encoded stones, encoded food supplements or any other inert material and a combination thereof.

It is a further object of the present invention to disclose the irradiated crystal as defined in any of the above, wherein the aqueous preparation is in the form of a lotion, mixture, compound, formulation, gel, cream, liquid infusion, spray, suspension, emulsion, emollient preparation, emollient moisturizer and any combination thereof.

It is a further object of the present invention to disclose the irradiated crystal as defined in any of the above, wherein the aqueous preparation is applied topically to the integuments and covering or support tissues of mammals, in human or veterinary fields.

### BRIEF DESCRIPTION OF THE DRAWINGS

In order to understand the invention and to see how it may be implemented in practice, a few preferred embodiments will now be described, by way of non-limiting example only, with reference to be accompanying drawings, in which:

FIGS. 1 and 2 illustrate a graph of the elastics properties of human skin of the product HYDRATANT SPF 15 (sample #1);

FIG. 3 illustrates a graph of the hydrating activity of the product HYDRATANT SPF 15 (sample #1);

FIG. 4 illustrates a graph of the Skin profilometry of the product HYDRATANT SPF 15 (sample #1);

FIGS. 5 and 6 illustrate a graph of the elastics properties of human skin of the product HYDRATANT SPF 15 (sample #2);

FIG. 7 illustrates a graph of the hydrating activity of the product HYDRATANT SPF 15 (sample #2); and

FIG. 8 illustrates a graph of the Skin profilometry of the product HYDRATANT SPF 15 (sample #2).

### DETAILED DESCRIPTION OF THE INVENTION

The following description is provided, alongside all chapters of the present invention, so as to enable any person skilled in the art to make use of the invention and sets forth the best modes contemplated by the inventor of carrying out this invention. Various modifications, however, is adapted to remain apparent to those skilled in the art, since the generic principles of the present invention have been defined specifically to provide means and method for the treatment of skin disorders.

The present invention provides a composition comprising at least one type of irradiated crystals and an aqueous preparation wherein the irradiated crystals are exposed to a changing electromagnetic field.

According to main aspects, the invention relates to preparations that are directed for external use or that are used topically.

It is herein acknowledged that the definition of a ‘composition’ or ‘cosmetic composition’ in the context of the present invention is any compound, substance or preparation intended to be placed in contact with the various parts of the human body i.e. skin tissue, dermis, epidermis, hypodermis, basement membrane, hair system, nails, lips, body organs, external genital organs or teeth. Furthermore, a cosmetic composition in the context of the present invention is that which is given in the regulations in this field (i.e. The General Product Safety 2005, The Cosmetic Products Safety Regulations 2008). The Regulations define a cosmetic product as any substance or preparation intended to be placed in contact with the various external parts of the human body (epidermis, hair system, nails, lips and external genital organs) or with the teeth and the mucous membranes of the oral cavity with a view exclusively or mainly to cleaning them, perfuming them, changing their appearance, correcting body odours, protecting them, or keeping them in good condition.

In another embodiment of the present invention, the composition may further be useful for the treatment of the skin, tissue, inner tissue and/or body organs or regions in a variety of medical conditions such as infection, inflammation, body fluid secretion such as focal hyperhidrosis and the like. The composition may further be used as an antibacterial or antimicrobial agent, antifungal agent, pain relief, stress, depression or anxiety, toxin treatment, oxidative stress, muscle relief, free radicals activity and the like.

In the context of the present invention, the composition can in particular carry a cosmetically effective active agent.

In the context of the present invention, the composition of the present invention may be used as a medicine, para-medicine product or as an intermediate product in cosmetic preparations, pharmaceutical compositions or food supplements.

According to a preferred embodiment, the compositions of the present invention comprise an aqueous preparation and at least one type of irradiated crystals. These crystals are exposed to a changing electromagnetic field. In a specific embodiment, the electromagnetic field is applied according to a predetermined protocol. The features of the aforementioned fields applied can be defined by: the frequency of repetition of the electromagnetic wave applied and the waveform; the intensity of the alternating field applied; the intensity of the electromagnetic field applied.

As used herein the term ‘aqueous preparation’ refers herein generally to a homogenous liquid preparation of any substance dissolved in water. More specifically, an aqueous preparation may refer to a preparation in the form of a lotion, mixture, compound, formulation, gel, cream, serum, liquid infusion, spray, suspension, emulsion, emollient preparation, emollient moisturiser and any combination thereof. For example an aqueous preparation in the context of the present invention may be an aqueous cream, which may be defined as a light, non-greasy moisturiser that is made from a mixture of emulsifying ointment (which contains paraffin oils) and water, with phenoxyethanol as an antimicrobial preservative. Furthermore the an aqueous preparation in the context of the present invention may be a body lotion, facial cream, lipstick, eye cream, soup, peeling, face/body makeup, serum, facial cleansing milk, skin toner, facial/body cleaner, eye/face/makeup remover and any product applied upon skin face and/or body.

The term ‘crystal’ or ‘smart crystal’ as used herein refers to a solid material or matrix whose constituent atoms, molecules, or ions are arranged in an ordered pattern extending in all three spatial dimensions. In the context of the present invention, the crystal is used a neutral and inert matrix that is exposed to the predetermined protocol of alternating electromagnetic fields. The crystals are preferably made of inert materials such as pearls, smart pearls e.g. having scientific pearls effect, silicon, sucrose, quartz, smart patches and any smart/intelligent particles or materials having a crystallized structure.

The term ‘about’ as used herein denotes ±25% of the defined amount or measure or value.

The term ‘encoded cosmetic composition’ or ‘encoded composition’ or ‘encoded cosmetic preparation’ as used herein refer to compositions or preparations comprising at least one type of crystals or crystal which has been exposed to a changing or alternating electromagnetic field. These encoded compositions are disclosed by the present invention to be effective as antiaging and useful in skin regeneration and recovery.

The term ‘smart crystal’, ‘smart pearls’, ‘smart patches’, ‘smart/intelligent particles’, ‘encoded gems’, ‘encoded stones’, ‘encoded food supplements’ and derivatives thereof as used herein are equivalent to components, dressing, food supplements, materials or matrices of the compositions comprising crystals, pearls, patches, bandages, splint, plaster, gauze, medical dressing or any particles or dressing characterized by a scientific effect thereby, have been irradiated or exposed to changing or alternating electromagnetic field. Furthermore, the encoded particles, material or matrices when interacting or admixed with any other material are with the ability to transfer radiation energy and further effect other materials. These encoded particles, dressing, materials, matrices or food supplements are disclosed by the present invention to be effective as antiaging preparations and promoters and useful in skin regeneration and recovery.

The term ‘GMP’ as used herein, refers to the Good Manufacturing Practices standards of the United States. In certain more specific embodiments, the final composition product is produced such that both the product and process for producing the product comply with GMP requirements.

Thus according to one embodiment, the present invention provides a composition comprising at least one type of smart crystals which are irradiated crystals and an aqueous preparation. In a core aspect of the invention the irradiated crystals are exposed to a changing electromagnetic field.

In accordance with another embodiment of the present invention, the changing electromagnetic field is applied according to a predetermined protocol. In certain aspects, the features of the applied electromagnetic fields (waveform, frequency, duty cycle, intensity) may depend on the type of result desired (regeneration, repair, growth), and on the various background conditions (type of tissue atrophy, type of substrate, time). The features of the fields applied may be given by: the frequency of repetition of the electromagnetic wave applied and the waveform; the intensity of the alternating electromagnetic field applied; the intensity of static magnetic and electric fields applied; and the application time.

In accordance with another embodiment of the present invention, the changing electromagnetic field oscillates at a frequency in the range of between about 0.1 and 1000 Hz.

In accordance with another embodiment of the present invention, the changing electromagnetic field oscillates at a frequency in the range of between about 0.2 and 200 Hz.

In accordance with another embodiment of the present invention, the changing electromagnetic field oscillates at a frequency in the range of between about 1 and 150 Hz.

In accordance with another embodiment of the present invention, the changing electromagnetic field is applied in increased frequencies.

In accordance with another embodiment of the present invention, the changing electromagnetic field is applied in a series of predetermined intensities in the range of 1 and 1000 μT.

In accordance with another embodiment of the present invention, the changing electromagnetic field is applied in a series of predetermined intensities in the range of 10 and 500 μT, preferably 5 to 200 μT.

In accordance with another embodiment of the present invention, the changing electromagnetic field is applied in a continuous waveform.

It is also within the scope of the present invention to provide the composition as described in any of the above, wherein the changing electromagnetic field is applied in a pulsed waveform.

It is also within the scope of the present invention to provide the composition as described in any of the above, wherein the crystal is made of sucrose, silicon, pearl, smart pearl quartz, smart particles, encoded gems, encoded stones, encoded food supplements or any other inert material and a combination thereof.

It is also within the scope of the present invention to provide the composition as described in any of the above, wherein the aqueous preparation is in the form of a lotion, mixture, compound, formulation, gel, cream, serum, liquid infusion, spray, suspension, emulsion, emollient preparation, emollient moisturiser and any combination thereof.

It is also within the scope of the present invention to provide the composition as described in any of the above, wherein the composition is adapted to improve or treat at least one medical parameter or condition in a mammal, preferably in a human.

It is also within the scope of the present invention to provide the composition as described in any of the above, wherein the composition is adapted to prevent at least one medical condition in a mammal, preferably in a human.

It is also within the scope of the present invention to provide the composition as described in any of the above, wherein the composition is adapted to stimulate the regeneration and/or the recovery of an organ, tissue or a system or a portion thereof in a mammal, preferably in a human.

It is also within the scope of the present invention to provide the composition as described in any of the above, for further preventing and treating laxity (sagging), rhytids (wrinkles), and photoaging, which includes erythema (redness), dyspigmentation (brown discolorations), solar elastosis (yellowing), keratoses (abnormal growths), and poor texture of the skin.

It is also within the scope of the present invention to provide the composition as described in any of the above, wherein the medical parameter or condition is selected from the group consisting of skin related parameters and/or disorders, recovery of an organ, tissue or system or a portion thereof, brain related parameters and any combination thereof.

It is also within the scope of the present invention to provide the composition as described in any of the above, wherein the skin related parameters and/or disorders are selected from the group consisting of trophism of the integumentary system, regeneration of skin cells, skin irritation, skin infection, skin inflammation, the effectiveness of the skin as a barrier, skin aging, skin pigmentation, allergy, dermatitis, allergic dermatitis, skin degeneration or atrophy and any combination thereof.

It is also within the scope of the present invention to provide the composition as described in any of the above, wherein the organ, tissue or system is selected from the group consisting of the lymph system, the endocrine system, the liver, skin, the integumentary system and any portion or combination thereof.

It is also within the scope of the present invention to provide the composition as described in any of the above, wherein the brain related parameters are selected from the group consisting of regulation and/or controlling of the muscle tonus center and/or motor control center of the cerebellum, the reflex centers, regulation or controlling of the posterior pituitary (Neurohypophysis) of the pituitary gland or hypophysis, regulation of the skin center in the brain and any combination thereof.

It is also within the scope of the present invention to provide the composition as described in any of the above, wherein the composition is applied topically to the integuments and covering or support tissues of mammals, in human or veterinary fields.

The present invention further discloses a process for the preparation of a composition for prevention, treatment, stimulation and/or recovery of at least a portion of a mammalian tissue or system, preferably the skin system, comprising the steps of: (a) providing an aqueous preparation; (b) providing at least one type of crystal; (c) exposing the at least one type of crystal to a changing electromagnetic field so as to obtain at least one type of an irradiated crystals; and (d) contacting the at least one type of irradiated crystals with the aqueous preparation thereby producing the composition for prevention, treating, and/or stimulating the recovery of the at least a portion of a mammalian tissue or system, preferably the skin system.

It is still within the scope of the present invention to provide the process as described in any of the above; comprising an additional step of exposing the at least one type of crystal to a changing electromagnetic field according to a predetermined protocol.

It is still within the scope of the present invention to provide the process as described in any of the above; comprising an additional step of exposing the at least one type of crystal to a changing electromagnetic field oscillating at a frequency in the range of between about 0.1 and 1000 Hz.

It is still within the scope of the present invention to provide the process as described in any of the above; comprising an additional step of exposing the at least one type of crystal to a changing electromagnetic field oscillating at a frequency in the range of between about 0.2 and 200 Hz.

It is still within the scope of the present invention to provide the process as described in any of the above; comprising an additional step of exposing the at least one type of crystal to a changing electromagnetic field oscillating at a frequency in the range of between about 1 and 150 Hz.

It is still within the scope of the present invention to provide the process as described in any of the above; comprising an additional step of exposing the at least one type of crystal to a changing electromagnetic field applied in increased frequencies.

It is still within the scope of the present invention to provide the process as described in any of the above, wherein the step of exposing the at least one type of crystal to a changing electromagnetic field is applied in a series of predetermined intensities in the range of 1 and 1000 Mt.

It is still within the scope of the present invention to provide the process as described in any of the above, wherein the step of exposing the at least one type of crystal to a changing electromagnetic field is applied in a series of predetermined intensities in the range of 10 and 500 μT, preferably 5 to 200 μT.

It is still within the scope of the present invention to provide the process as described in any of the above, wherein the step of exposing the at least one type of crystal to a changing electromagnetic field is applied in a continuous waveform.

It is still within the scope of the present invention to provide the process as described in any of the above, wherein the step of exposing the at least one type of crystal to a changing electromagnetic field is applied in a pulsed waveform.

It is still within the scope of the present invention to provide the process as described in any of the above; comprising an additional step of providing at least one type of crystal made of sucrose, silicon, pearl, smart pearl, quartz, smart patches or any other inert material and a combination thereof.

It is still within the scope of the present invention to provide the process as described in any of the above; comprising an additional step of providing an aqueous preparation in the form of a lotion, mixture, compound, formulation, gel, cream, liquid infusion, spray, suspension, emulsion, emollient preparation, emollient moisturiser and any combination thereof.

It is still within the scope of the present invention to provide the process as described in any of the above, wherein the process complies with GMP reproducibility standards.

It is still within the scope of the present invention to provide the process as described in any of the above, wherein the final composition product meets the standard GMP requirements.

The present invention further provides a method for prevention, treatment, stimulation and/or recovery of at least a portion of a mammalian tissue or system, preferably the skin system, wherein the method comprising the steps of: (a) providing a composition comprising at least one type of irradiated crystals and an aqueous preparation wherein said irradiated crystals are exposed to a changing electromagnetic field, and (b) applying the composition topically to at least a portion of the integuments, covering or support tissues of mammals so as to prevent, treat, and/or stimulate the recovery of the at least a portion of a mammalian tissue or system, preferably the skin system.

It is still within the scope of the present invention to provide the method as described in any of the above comprising an additional step of applying the changing electromagnetic field according to a predetermined protocol.

It is still within the scope of the present invention to provide the method as described in any of the above comprising an additional step of exposing the at least one type of crystal to a changing electromagnetic field oscillating at a frequency in the range of between about 0.1 and 1000 Hz.

It is still within the scope of the present invention to provide the method as described in any of the above comprising an additional step of exposing the at least one type of crystal to a changing electromagnetic field oscillating at a frequency in the range of between about 0.2 and 200 Hz.

It is still within the scope of the present invention to provide the method as described in any of the above comprising an additional step of exposing the at least one type of crystal to a changing electromagnetic field oscillating at a frequency in the range of between about 1 and 150 Hz.

It is still within the scope of the present invention to provide the method as described in any of the above comprising an additional step of applying the changing electromagnetic field in increased frequencies.

It is still within the scope of the present invention to provide the method as described in any of the above comprising an additional step of applying the changing electromagnetic field in a series of predetermined intensities in the range of 1 and 1000 μT.

It is still within the scope of the present invention to provide the method as described in any of the above comprising an additional step of applying the changing electromagnetic field in a series of predetermined intensities in the range of 10 and 500 μT, preferably 5 to 200 μT.

It is still within the scope of the present invention to provide the method as described in any of the above comprising an additional step of applying the changing electromagnetic field in a continuous waveform.

It is still within the scope of the present invention to provide the method as described in any of the above comprising an additional step of applying the changing electromagnetic field in a pulsed waveform.

It is still within the scope of the present invention to provide the method as described in any of the above comprising an additional step of providing the crystal made of sucrose, silicon, pearl, smart pearl, quartz, smart particles, encoded gems, encoded stones, encoded food supplements or any other inert material and a combination thereof.

It is still within the scope of the present invention to provide the method as described in any of the above comprising an additional step of preparing the aqueous preparation is in the form of a lotion, mixture, compound, formulation, gel, cream, liquid infusion, spray, suspension, emulsion, emollient preparation, emollient moisturiser and any combination thereof.

It is still within the scope of the present invention to provide the method as described in any of the above comprising an additional step of improving or treating at least one medical parameter or condition in a mammal, preferably in a human.

It is still within the scope of the present invention to provide the method as described in any of the above comprising an additional step of preventing at least one medical condition in a mammal, preferably in a human.

It is still within the scope of the present invention to provide the method as described in any of the above comprising an additional step of stimulating the regeneration and/or the recovery of an organ, tissue or a system or a portion thereof in a mammal, preferably in a human.

It is still within the scope of the present invention to provide the method as described in any of the above comprising an additional step of selecting the medical parameter or condition from the group consisting of skin related parameters and/or disorders, recovery of an organ, tissue or system or a portion thereof, brain related parameters and any combination thereof.

It is still within the scope of the present invention to provide the method as described in any of the above comprising an additional step of selecting the skin related parameters and/or disorders from the group consisting of trophism of the integumentary system, regeneration of skin cells, skin irritation, skin infection, skin inflammation, the effectiveness of the skin as a barrier, skin aging, skin pigmentation, allergy, dermatitis, allergic dermatitis, skin degeneration or atrophy and any combination thereof.

It is still within the scope of the present invention to provide the method as described in any of the above comprising an additional step of selecting the organ, tissue or system from the group consisting of the lymph system, the endocrine system, the liver, skin, the integumentary system and any portion or combination thereof.

It is still within the scope of the present invention to provide the method as described in any of the above comprising an additional step of selecting the brain related parameters from the group consisting of regulation and/or controlling of the muscle tonus center and/or motor control center of the cerebellum, the reflex centers, regulation or controlling of the posterior pituitary (Neurohypophysis) of the pituitary gland or hypophysis, regulation of the skin center in the brain and any combination thereof.

It is still within the scope of the present invention to provide the method as described in any of the above comprising an additional step of applying the composition topically to the integuments and covering or support tissues of mammals, in human or veterinary fields.

The present invention further provides an irradiated crystal activated by exposure to a changing electromagnetic field, for topical use.

The present invention further provides an irradiated crystal activated by exposure to a changing electromagnetic field, wherein the irradiated crystal is useful in cosmetic preparations.

It is within the scope of the present invention to provide the aforementioned irradiated crystal, wherein the irradiated crystal is useful in aqueous cosmetic preparations.

It is within the scope of the present invention to provide the aforementioned irradiated crystal, wherein the changing electromagnetic field is applied according to a predetermined protocol.

It is within the scope of the present invention to provide the aforementioned The irradiated crystal, wherein the changing electromagnetic field oscillates at a frequency in the range of between about 0.1 and 1000 Hz.

It is within the scope of the present invention to provide the aforementioned irradiated crystal, wherein the changing electromagnetic field oscillates at a frequency in the range of between about 0.2 and 200 Hz.

It is within the scope of the present invention to provide the aforementioned irradiated crystal, wherein the changing electromagnetic field oscillates at a frequency in the range of between about 1 and 150 Hz.

It is within the scope of the present invention to provide the aforementioned irradiated crystal, wherein the changing electromagnetic field is applied in increased frequencies.

It is within the scope of the present invention to provide the aforementioned irradiated crystal, wherein the changing electromagnetic field is applied in a series of predetermined intensities in the range of 1 and 1000 μT.

It is within the scope of the present invention to provide the aforementioned irradiated crystal, wherein the changing electromagnetic field is applied in a series of predetermined intensities in the range of 10 and 500 μT, preferably 5 to 200 μT.

It is within the scope of the present invention to provide the aforementioned irradiated crystal, wherein the changing electromagnetic field is applied in a continuous waveform.

It is within the scope of the present invention to provide the aforementioned irradiated crystal, wherein the changing electromagnetic field is applied in a pulsed waveform.

It is within the scope of the present invention to provide the aforementioned irradiated crystal, wherein the crystal is selected form the group consisting of sucrose, silicon, pearl, smart pearl quartz, smart patches, smart pearls, smart patches, smart particles, encoded gems, encoded stones, encoded food supplements or any other inert material and a combination thereof.

It is within the scope of the present invention to provide the aforementioned irradiated crystal, wherein the aqueous preparation is in the form of a lotion, mixture, compound, formulation, gel, cream, liquid infusion, spray, suspension, emulsion, emollient preparation, emollient moisturiser and any combination thereof.

It is within the scope of the present invention to provide the aforementioned irradiated crystal, wherein the aqueous preparation is applied topically to the integuments and covering or support tissues of mammals, in human or veterinary fields.

In order to understand the invention and to see how it may be implemented in practice, a plurality of preferred embodiments will now be described, by way of non-limiting example only, with reference to the following examples.

### EXAMPLES

### Example 1

### Preparation of Irradiated Crystals According to Predetermined Protocols

According to one embodiment, smart crystal which are irradiated crystals have been prepared by exposing at least one type of crystals to a changing electromagnetic field. According to a specific embodiment, the at least one type of crystals are exposed to a device capable of generating electromagnetic fields. The features of the electromagnetic fields, such as waveform, frequency, intensity, duty cycle and static electric and magnetic fields possibly added to the changing electromagnetic fields are determined by the type of result desired (i.e. regeneration, growth or repair) and the type of targeted tissue or organ. According to this embodiment, the features of the electromagnetic fields applied are predetermined and are directed to treat or affect a specific medical parameter or condition, preferably associated with the skin.

According to a further embodiment, the crystals are made of inert materials and are used as inert matrices encapsulating the predetermined electromagnetic frequencies. Examples of such inert materials used in the current experiment include sucrose and smart pearls.

In a further embodiment, the applied electromagnetic field oscillates at sequential alternating frequencies in the range of about 0.2 Hz to about 1000 Hz and more preferably in the range of 0.2 Hz to about 200 Hz. Examples of such irradiation protocols and their potential affected targeted medical parameter or condition are provided in Table 1. It is herein emphasized that the protocols are effective in both prevention and treatment or control or regulation of the target medical parameter or condition.

TABLE 1
Irradiation protocols and potential targeted
medical parameter or condition

| Irradiation | |
|---|---|
| protocol (*) | Target medical parameter or condition |
| A | Regulation and maintenance of the integumentary |
| | system- control center in the brain |
| | Skin cells regeneration |
| | Skin irritation |
| | Skin infections |
| B | The effectiveness of the skin as a barrier |
| | Anti allergy |
| | Skin allergy (i.e. caused by chemicals, animals, |
| | foodstuff) |
| | Allergic dermatitis |
| C | Regulation of the muscle tonus center and/or motor |
| | control center of the cerebellum and the reflex |
| | centers |
| | Regulation and maintenance of the posterior |
| | pituitary (Neurohypophysis) of the pituitary gland |
| | or hypophysis in the brain |
| D | Stimulation of skin regeneration |
| | Decreasing skin degeneration |
| | Decreasing skin aging |
| | Decreasing skin pigmentation (red and white |
| | pigments) |

* Irradiation protocol index (sequential applied frequencies in Hertz):
A- 3.20, 4.87, 5.80, 5.89, 7.00, 7.25, 25.00, 56.25, 62.52, 64.00, 90.00, 91.25, 95.60
B- 17.00, 18.50, 19.62, 22.80, 25.85, 32.00, 42.80, 45.70, 52.80, 72.43, 81.40, 102.30, 117.28
C- 23.80, 28.60, 33.90, 48.50, 54.00, 55.90, 114.80, 144.60, 154.70
D- 0.24, 3.87, 3.95, 8.44, 35.50, 49.70, 67.52, 74.30, 81.12, 94.80, 97.65, 100, 105.52, 116.70

### Example 2

### The Effect of the Compositions of the Present Invention on Tested Organs, Parameters and Systems in the Human Body

Cosmetic compositions containing at least one type of irradiated crystals exposed to changing electromagnetic fields according to the protocols described in example 1 were used. These compositions are herein used “coded cosmetic compositions”. The irradiated crystals were incorporated into cosmetic preparations such as hand cream, body cream and facial cream. Examples of cosmetic preparations used in the present invention include, but are not limited to cream and serum named “Crystal Youth”, Spiral Zen System”, “Lavilin Bio Balance”, “Ambiance” and “Love Line Bio Balance”, “QL” of Hlavin Industries.

About ten tested subjects were exposed to the cosmetic compositions of the present invention, specifically to hand creams and body creams comprising irradiated crystals. The treated subjects were exposed to the cosmetic compositions about twice a week for about a month and a half (43 days).

The tested parameters included monitoring of organs, systems or medical conditions such as: the lymph system, allergy (allergic reactions), the endocrine system, the liver and the skin (integumentary system). These parameters were scored before and after using irradiated hand cream and body cream comprising crystals irradiated according to protocols described in example I above. The maximum score for each organ or system was 50-60 points. Each tested subject was scored for all the above described parameters. The results obtained in the experiment are described herein below:

1. Subject A

Age: 56

Irradiation protocol used: A

TABLE 2
Scoring results of subject A

| Tested organ, system | | Score after 43 |
|---|---|---|
| or condition | Score before treatment | days of treatment |
| Lymph system | 47 | 50 |
| Allergy | 60 | 55-60 |
| Endocrine system | 43 | 45 |
| Liver | 50 | 50 |
| Skin | 36 | 55 |

2. Subject B

Age: 53

Irradiation protocol used: A

TABLE 3
Scoring results of subject B

| Tested organ, system | | Score after 43 |
|---|---|---|
| or condition | Score before treatment | days of treatment |
| Lymph system | 30 | 50 |
| Allergy | 20 | 38 |
| Endocrine system | 20 | 30 |
| Liver | 11 | 50 |
| Skin | 28 | 52 |

3. Subject C

Age: 37

Irradiation protocol used: A+B

TABLE 4
Scoring results of subject C

| Tested organ, system | | Score after 43 |
|---|---|---|
| or condition | Score before treatment | days of treatment |
| Lymph system | 55 | 48 |
| Allergy | 35-40 | 52 |
| Endocrine system | 45 | 48 |
| Liver | 48 | 40 |
| Skin | 38 | 55 |

4. Subject D

Age: 57

Irradiation protocol used: A

TABLE 5
Scoring results of subject D

| Tested organ, system | | Score after 43 |
|---|---|---|
| or condition | Score before treatment | days of treatment |
| Lymph system | 28 | 42 |
| Allergy | 20 | 38 |
| Endocrine system | 15 | 35 |
| Liver | 18 | 40 |
| Skin | 28 | 48 |

5. Subject E

Age: 62

Irradiation protocol used: A

TABLE 6
Scoring results of subject E

| Tested organ, system | | Score after 43 |
|---|---|---|
| or condition | Score before treatment | days of treatment |
| Lymph system | 10 | 25 |
| Allergy | 15 | 25 |
| Endocrine system | 45 | 35 |
| Liver | 19 | 48 |
| Skin | 28 | 48 |

6. Subject F

Age: 35

Irradiation protocol used: A

TABLE 7
Scoring results of subject F

| Tested organ, system | | Score after 43 |
|---|---|---|
| or condition | Score before treatment | days of treatment |
| Lymph system | 18 | 48 |
| Allergy | 38 | 35 |
| Endocrine system | 16 | 40 |
| Liver | 15 | 35 |
| Skin | 25 | 30 |

7. Subject G

Age: 44

Irradiation protocol used: A

TABLE 8
Scoring results of subject G

| Tested organ, system or | | Score after 43 days of |
|---|---|---|
| condition | Score before treatment | treatment |
| Lymph system | 55 | 55 |
| Allergy | 38 | 55 |
| Endocrine system | 50 | 52 |
| Liver | 30 | 30 |
| Skin | 35 | 48 |

8. Subject H

Age: 35

Irradiation protocol used: A

TABLE 9
Scoring results of subject H

| Tested organ, system | | Score after 43 |
|---|---|---|
| or condition | Score before treatment | days of treatment |
| Lymph system | 40 | 25 |
| Allergy | 15 | 30 |
| Endocrine system | 30 | 25 |
| Liver | 40 | 38 |
| Skin | 25 | 50 |

9. Subject I

Age: 42

Irradiation protocol used: A

TABLE 10
Scoring results of subject I

| Tested organ, system | | Score after 43 |
|---|---|---|
| or condition | Score before treatment | days of treatment |
| Lymph system | 28 | 40 |
| Allergy | 30 | 38 |
| Endocrine system | 39 | 40 |
| Liver | 50 | 60 |
| Skin | 38 | 55 |

10. Subject J

Age: 56

Irradiation protocol used: A

TABLE 11
Scoring results of subject J

| Tested organ, system or | | Score after 43 days of |
|---|---|---|
| condition | Score before treatment | treatment |
| Lymph system | 10 | 45 |
| Allergy | 28 | 32 |
| Endocrine system | 28 | 40 |
| Liver | 38 | 35 |
| Skin | 19 | 40 |

The results described above clearly show that the compositions of the present invention are effective and efficacious in all the tested aspects. Increased score results were observed with respect to the lymph system, allergy symptoms, the endocrine system, the liver and the skin, after treatment with the encoded compositions of the present invention. The most effective results were shown for the skin system with an average improvement of about 64% in the score value after treatment with the compositions of the present invention as compared to the control. To conclude, the compositions of the present invention, comprising preparations such as hand or body cream and irradiated crystals exposed to changing electromagnetic fields (i.e. irradiation protocols A, B, C, D and their combinations) are herein shown to be useful and efficacious in improving parameters related to major systems in the body such as the lymph systems, allergy symptoms, the endocrine system, the liver and especially the skin.

### Example 3

### The Effect of the Compositions of the Present Invention on Facial Skin

The compositions of the present invention, comprising aqueous cosmetic preparations and irradiated crystals exposed to changing electromagnetic fields were tested for their effectively with respect to dermal parameters and more specifically with respect to facial skin parameters. Irradiated crystals were introduced into peeling cosmetic compositions and collagen mask cosmetic products to produce the cosmetic compositions of the present invention. The irradiated crystals were preferably made of pearls or smart pearls having scientific pearls effect and were exposed to irradiation protocols (A, B, C or D) as described above and their combinations. The effect of the peeling and collagen mask encoded compositions of the present invention on five tested subjects is described in Tables 12 and 13 below.

TABLE 12
Scoring results of encoded peeling compositions

| | Subject 1 | Subject 2 | Subject 3 | Subject 4 | Subject 5 |
|---|---|---|---|---|---|
| Control | 39 | 41 | 65 | 43 | 42 |
| Peeling | 42 | 41 | 59 | 43 | 42 |
| composition | | | | | |
| Peeling | 49 | 41 | 50 | 45 | 42 |
| composi- | | | | | |
| tion + | | | | | |
| protocol C | | | | | |
| Peeling | 50 | 43 | 54 | 45 | 42 |
| composi- | | | | | |
| tion + | | | | | |
| protocol | | | | | |
| (B + C) | | | | | |

TABLE 13
Scoring results of encoded collagen mask compositions

| | Subject 1 | Subject 2 | Subject 3 | Subject 4 | Subject 5 |
|---|---|---|---|---|---|
| Control | 39 | 41 | 65 | 42 | 86 |
| Collagen | 41 | 49 | 50 | 41 | 66 |
| mask | | | | | |
| Collagen | 50 | 49 | 50 | 50 | 52 |
| mask + | | | | | |
| protocol A | | | | | |
| Collagen | 46 | 50 | 50 | 50 | 50 |
| mask + | | | | | |
| protocol B | | | | | |
| Collagen | 46 | 50 | 50 | 49 | 54 |
| mask + | | | | | |
| protocol D | | | | | |
| Collagen | 50 | 50 | 50 | 49 | 56 |
| mask + | | | | | |
| protocol | | | | | |
| (A + B + D) | | | | | |

The results described in Tables 12 and 13 demonstrate that by using the encoded compositions of the present invention, skin regeneration and recovery can be achieved. The preparations comprising irradiated crystals exposed to predetermined electromagnetic field oscillations such as the irradiation protocols A, B, C, D and their combinations are herein shown to be useful in stimulating and/or synergistically enhancing the efficacy of dermal or skin products or compositions.

### Example 4

### Sample #1

### Improvement of the Elastics Properties of Human Skin

The cosmetic product HYDRATANT SPF 15 (#1) was prepared and sampled.

HYDRATANT SPF 15 Ingredients: Aqua (Water), Octocrylene, Stearyl Alcohol, Phenylbenzimidazole Sulfonic Acid, C12-15 Alkyl Benzoate, Ethylhexyl Salicylate, Butyl Methoxydibenzoylmethane, Cetearyl Olivate, Sorbitan Olivate, Peg-40 Stearate, Cetyl Phosphate, Disteardimonium Hectorite and Propylene Carbonate, Sorbitan Stearate, Titanium Dioxide, Hydrated Silica, Dimethicone/Methicone Copolymer, Aluminum Hydroxide, Phenoxyethanol & Ethylhexylglycerin, 1,2-Hexanediol, Caprylyl Glycol, Sodium Hydroxide, Sorbitan Palmitate, Cetyl Palmitate, Squalene, Cyclopentasiloxane, Nymphaea Coerulea (Blue Lotus) Flower Extract, Hamamelis Virginiana (Witch Hazel) Extract, Allantoin, Isohexadecane, P.V.P, Lactose, Milk Protein, Bifida Ferment Lysate Sucrose, Cyclomethicone, Fragrance, Disodium Lauriminodipropionate Tocopheryl Phosphates, Xylitylglucoside and xylitol.

According to one embodiment, the HYDRATANT SPF 15 product comprising irradiated crystals. The irradiated crystals have been prepared by exposing at least one type of crystals to a changing electromagnetic field. The irradiated crystals have been prepared by exposing at least one type of crystals to a changing electromagnetic field. According to a specific embodiment, the at least one type of crystals are exposed to a device capable of generating electromagnetic fields. In one embodiment, such a device comprises coils that may be programmed, i.e. by an electronic computerized system to generate changing electromagnetic fields. The features of the electromagnetic fields, such as waveform, frequency, intensity, duty cycle and static electric and magnetic fields possibly added to the changing electromagnetic fields are determined by the type of result desired (i.e. regeneration, growth or repair) and the type of targeted tissue or organ.

At least one type of irradiated crystals of HYDRATANT SPF 15 (such as Bifida Ferment Lysate Sucrose or/and Silica) exposed to changing electromagnetic fields according to the following frequencies protocols index (sequential applied frequencies in Hertz):

A—3.20, 4.87, 5.80, 5.89, 7.00, 7.25, 25.00, 56.25, 62.52, 64.00, 90.00, 91.25, 95.60

B—17.00, 18.50, 19.62, 22.80, 25.85, 32.00, 42.80, 45.70, 52.80, 72.43, 81.40, 102.30, 117.28

C—23.80, 28.60, 33.90, 48.50, 54.00, 55.90, 114.80, 144.60, 154.70

D—0.24, 3.87, 3.95, 8.44, 35.50, 49.70, 67.52, 74.30, 81.12, 94.80, 97.65, 100, 105.52, 116.70

The HYDRATANT SPF 15 comprising the irradiated crystals (sample #1 and #2) was sampled and run several times and irradiated according to protocols A, B, C, D and a combination thereof.

The same HYDRATANT SPF 15 product comprising irradiated crystals (which were exposed to changing electromagnetic fields) was tested and is presented in Examples 4, 5, 6, 7, 8 and 9.

The cosmetic product HYDRATANT SPF 15 (#1) was applied on the intact skin of one inner forearm of 20 female volunteers in the age of 30 to 63, according to the recommended use directions (two applications per day, morning and evening, for 4 consecutive weeks).

A skin area chosen on the upper arm (deltoid) was not treated and served as control. In order to evaluate the improvement of the elastic properties of the outer layers of the skin, instrumental measurements of the skin elasticity were performed at the following times: before application (Tₒ) and at the end of the test period (T_{f}), on the treated area and on the control area (Not tested).

At the end of the period (T_{f}) the final measurements were carried out about 4 hours after the last product application.

### Principle of Method

Instrumental measurements are performed by Cutometer MPA 580. This instrument measures mechanical properties of the skin, such as the level of elasticity and extensibility, expression of skin tone and correlated with dermis and epidermis and functionality.

The principle of measurement consists in the suction of the test skin area (approx. 3 mm), by the defined pressure applied through the circular hole (2 mm in diameter) of a probe. This measuring probe was attached to the main unit by a cable for data transmission and a cable for air transport.

The skin elasticity reflects the potential retraction skin capacity (elastic retraction) after a suction/elongation, the skin extensibility shoes the level of skin extension after a suction. The product is applied for 4 consecutive weeks, on the test area of the treated forearm twice a day (morning and evening).

Skin elasticity was measured by Cutimeter MPA 580. This instrument measures the elastic properties of skin based on the principle of suction/elongation.

A mode with a low and constant level of pressure is adopted: the cutis is sucked into the probe with a uniform vacuum-operated for a specific time, than returns to its original position when the pressure is released.

The suction time adopted is 1 sec;

The release time adopted is 1 sec;

The pressure adopted is 350 mb.

The measurement is repeated for 3 consecutive cycles.

The computerized program was used which allows the calculation for the following parameters:

a) immediate distension=UE

b) delayed distention=Uv

c) immediate retraction=Ur

d) final distension=Uf

Two relative parameters indicative of mechanical properties of the skin are then calculated.

The viscoelastic to elastic ratio (Uv/UE): increasing values of Uv/Ue indicates an increasing viscoelastic portion of the deformation process and a relative decrease of the elastic portion. A decrease of the parameter reflects a general improvement in the elastic properties of the skin.

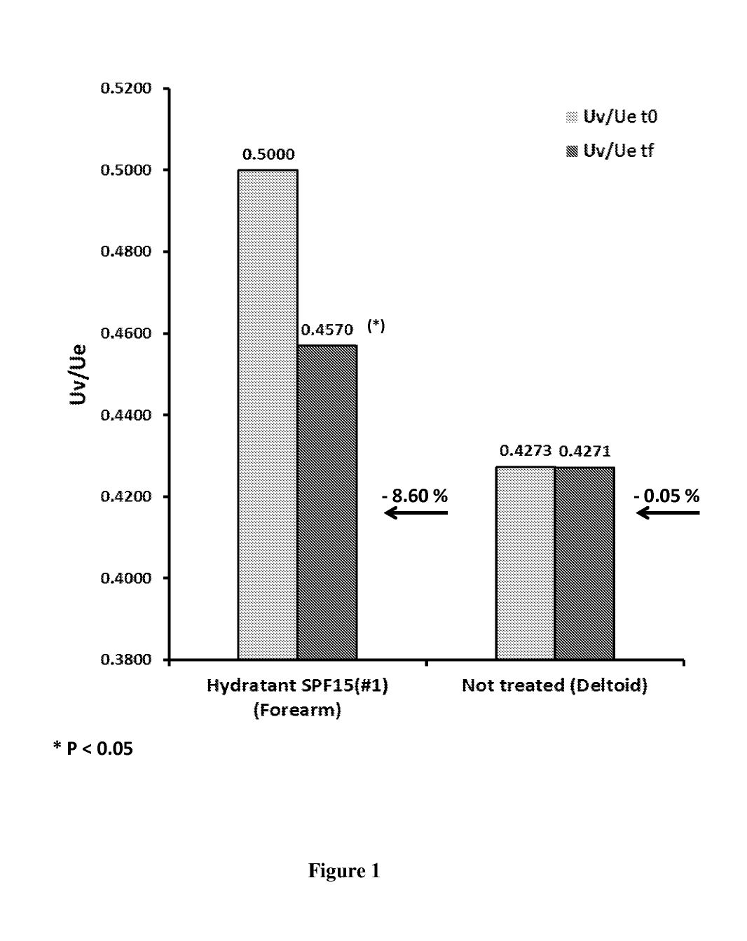

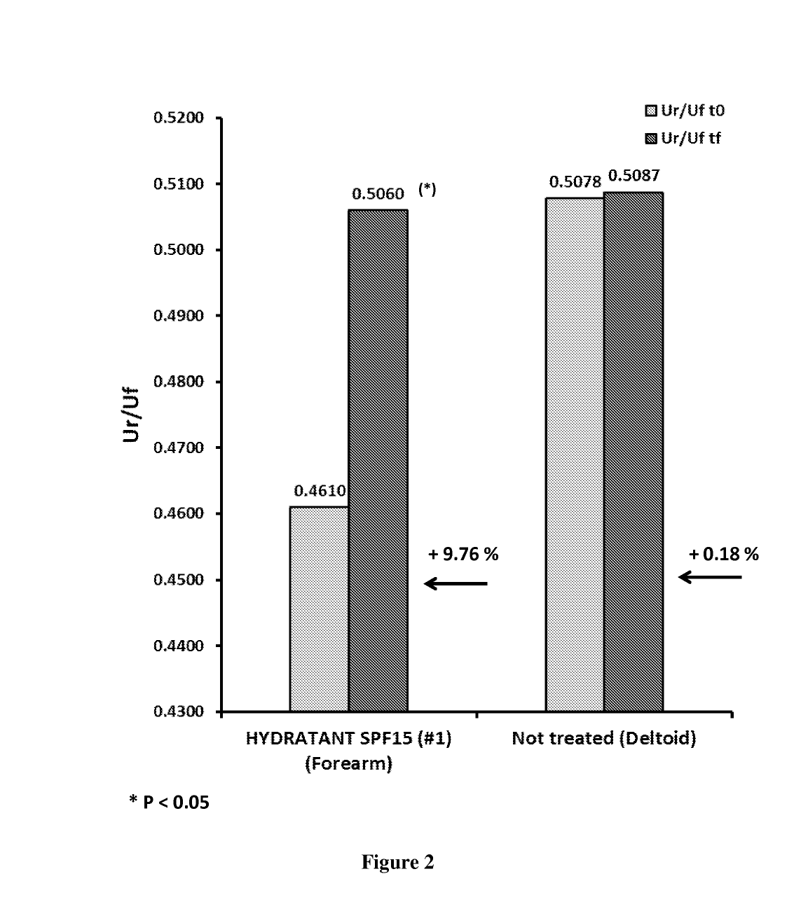

Biologic elasticity (Ur/Uf): it shows the ability of the skin to regain its initial position after deformation. A value of 1 would indicate 100% elasticity; an increase of this parameter reflects a general improvement in the elastic properties of the skin. The mean values measured in treated and control area are illustrated graphically. The data obtained are statistically analyzed and results are summarized in table 14, 15, 16 below and graphical representations enclosed to the test results (see FIG. 1 and FIG. 2). Statistical evaluation of final measurements against baseline values is performed by two tail t-test for comparison of the means.

A value of p≦0.05 is considered statistically significant.

TABLE 14
PRODUCT: HYDRATANT SPF 15 (# 1)
Tab. 1: TREATED AREA - Uv/Ue and Ur/Uf: values before product
application (T0) and after 4 consecutive weeks of product application (Tf).

| | First | | | Uv/Ue | Uv/Ue | Ur/Uf | Ur/Uf |
|---|---|---|---|---|---|---|---|
| # Vol | letter | Sex | AGE | T₀ | T_{f} | T₀ | T_{f} |
| 1 | T. S. | F | 51 | 0.262 | 0.230 | 0.508 | 0.559 |
| 2 | M. M. | F | 49 | 0.458 | 0.407 | 0.387 | 0.409 |
| 3 | M. S. | F | 36 | 0.474 | 0.456 | 0.472 | 0.528 |
| 4 | B. L. | F | 30 | 0.498 | 0.510 | 0.572 | 0.590 |
| 5 | A. R. | F | 53 | 0.695 | 0.628 | 0.414 | 0.452 |
| 6 | M. L. | F | 45 | 0.480 | 0.455 | 0.429 | 0.496 |
| 7 | C. A. | F | 47 | 0.698 | 0.631 | 0.520 | 0.605 |
| 8 | P. L. | F | 46 | 0.280 | 0.254 | 0.524 | 0.598 |
| 9 | M. N. | F | 53 | 0.464 | 0.403 | 0.379 | 0.444 |
| 10 | R. L. | F | 50 | 0.564 | 0.536 | 0.509 | 0.526 |
| 11 | C. L. | F | 46 | 0.463 | 0.427 | 0.496 | 0.510 |
| 12 | D. P. | F | 66 | 0.620 | 0.576 | 0.275 | 0.305 |
| 13 | R. L. | F | 64 | 0.284 | 0.245 | 0.500 | 0.560 |
| 14 | C. N. | F | 42 | 0.580 | 0.520 | 0.482 | 0.517 |
| 15 | M. C. | F | 45 | 0.249 | 0.213 | 0.510 | 0.536 |
| 16 | E. A. | F | 53 | 0.537 | 0.473 | 0.477 | 0.535 |
| 17 | M. E. | F | 31 | 0.601 | 0.548 | 0.488 | 0.563 |
| 18 | M. L. | F | 62 | 0.639 | 0.581 | 0.356 | 0.393 |
| 19 | T. L. | F | 58 | 0.462 | 0.393 | 0.435 | 0.464 |
| 20 | V. P. | F | 58 | 0.690 | 0.656 | 0.492 | 0.539 |

TABLE 15
PRODUCT: HYDRATANT SPF 15 (# 1)
CONTROL AREA (Not Treated) - Uv/Ue and Ur/Uf: values before (T0)
and after 4 consecutive weeks of product application (Tf).

| | First | | | Uv/Ue | Uv/Ue | Ur/Uf | Ur/Uf |
|---|---|---|---|---|---|---|---|
| # Vol | letter | Sex | AGE | T₀ | T_{f} | T₀ | T_{f} |
| 1 | T. S. | F | 51 | 0.251 | 0.258 | 0.574 | 0.599 |
| 2 | M. M. | F | 49 | 0.491 | 0.502 | 0.514 | 0.526 |
| 3 | M. S. | F | 36 | 0.346 | 0.331 | 0.478 | 0.478 |
| 4 | B. L. | F | 30 | 0.455 | 0.458 | 0.576 | 0.562 |
| 5 | A. R. | F | 53 | 0.549 | 0.544 | 0.553 | 0.513 |
| 6 | M. L. | F | 45 | 0.462 | 0.460 | 0.467 | 0.480 |
| 7 | C. A. | F | 47 | 0.593 | 0.593 | 0.452 | 0.445 |
| 8 | P. L. | F | 46 | 0.282 | 0.289 | 0.547 | 0.595 |
| 9 | M. N. | F | 53 | 0.332 | 0.325 | 0.518 | 0.519 |
| 10 | R. L. | F | 50 | 0.585 | 0.579 | 0.470 | 0.480 |
| 11 | C. L. | F | 46 | 0.439 | 0.439 | 0.591 | 0.599 |
| 12 | D. P. | F | 66 | 0.479 | 0.478 | 0.388 | 0.372 |
| 13 | R. L. | F | 64 | 0.524 | 0.526 | 0.480 | 0.482 |
| 14 | C. N. | F | 42 | 0.225 | 0.230 | 0.534 | 0.532 |
| 15 | M. C. | F | 45 | 0.237 | 0.237 | 0.621 | 0.594 |
| 16 | E. A. | F | 53 | 0.301 | 0.303 | 0.512 | 0.516 |
| 17 | M. E. | F | 31 | 0.553 | 0.553 | 0.481 | 0.485 |
| 18 | M. L. | F | 62 | 0.429 | 0.412 | 0.349 | 0.342 |
| 19 | T. L. | F | 58 | 0.459 | 0.452 | 0.512 | 0.515 |
| 20 | V. P. | F | 58 | 0.554 | 0.574 | 0.539 | 0.541 |

TABLE 16
PRODUCT: HYDRATANT SPF 15 (# 1)
Uv/Ue and Ur/Uf variations in the mean parameters levels describing
the elastic properties of the skin before (T0) and after 4 consecutive
weeks of treatment (Tf)

| | | | | | % |
|---|---|---|---|---|---|
| | | T₀ | Tf | p | variation |
| Uv/Ue | MEAN ± | 0.500 ± 0.143 | 0.457 ± 0.137 | <0.05 (*) | −8.60 |
| HYDRATANT SPF 15 (# 1) | S.D. | | | | |
| (Forearm) | | | | | |
| Uv/Ue | MEAN ± | 0.4273 ± 0.121 | 0.4271 ± 0.121 | >0.05 | −0.05 |
| CONTROL AREA | S.D. | | | | |
| (not treated - Deltoid)) | | | | | |
| Ur/Uf | MEAN ± | 0.461 ± 0.070 | 0.506 ± 0.079 | <0.05 (*) | +9.76 |
| HYDRATANT SPF 15 (# 1) | S.D. | | | | |
| (Forearm) | | | | | |
| Ur/Uf | MEAN ± | 0.5078 ± 0.066 | 0.5087 ± 0.069 | >0.05 | +0.18 |
| CONTROL AREA | S.D. | | | | |
| (not treated - Deltoid) | | | | | |

(*) a value of p ≦ 0.05 is considered statistically significant

The parameters describing the elastic properties of the outer layers of the skin were evaluated by instrumental measurements (Cutometer MPA 580 Courage & Khazaka). Measurements were taken before (T₀) and after the test period (T_{f}), both on the test area and the control (not treated area).

At the end of the test period (T_{f}) the final measurements were carries out about 4 hours after the last product application.

The following variations were observed in the parameters taken into consideration, as compared to the baseline (T₀):

The viscoelastic to elastic ratio (Uv/Ur): increasing values of Uv/Ur indicates an increasing viscoelastic portion of the deformation process and a relative decrease of the elastic portion. A decrease of this parameter reflects a general improvement in the elastic properties of the skin.

Biologic elasticity (Ur/Uf): shows the ability of the skin to regain its initial position after deformation. A value of 1 would indicate 100% elasticity. An increase of this parameter reflected a general improvement in the elastic properties of the skin.

It can therefore be concluded that the cosmetic product HYDRATANT SPF 15 (#1), applied in the adopted test condition, appears to be active in improving the elastic properties of the outer layer of the skin: skin deformability appeared significantly reduced and skin elasticity appeared significantly increased after 4 consecutive weeks of product application.

The following variations were observed in the parameters taken into consideration, as compared to the baseline (Tₒ):

Viscoelastic to elastic ratio (Uv/Ue): at the end of the application period the mean value of the parameter showed a decrease of about 8.60% (p<0.05) in the treated area, and a decrease of about 0.05% in the control area (p>0.05).

Decreasing values of Uv/Ue indicate a decreasing viscoelastic portion of the deformation process and a relative increase of the elastic portion (a decrease of this parameter illustrates a general improvement of skin elastic properties).

Biologic Elasticity (Ur/Uf): at the end of the application period, the mean value of this parameter showed an increase of about 9.76% (p<0.05) in the treated area, and an increase of about 0.18% in the control area (p>0.05).

This parameter shows the ability of the skin to regain its initial position after deformation (an increase of this parameter is indicative of a general improvement of skin elastic properties).

The variation observed in these two parameters show a global improvement in the elastic properties of the outer layers of the skin, following the application of the cosmetic product HYDRATANT SPF 15 (#1).

In the control area (not treated) the variations in the skin elastic properties were not statistically significant as compared to the baseline (p>0.05).

### Example 5

### Sample #1

### Hydrating Activity

The hydrating activity of the cosmetic product HYDRATANT SPF 15 (#1) was evaluated after a period of 4 consecutive weeks of application on a panel constituted of 20 volunteers, female, aged 20-70 (two applications per day. Morning and evening, on the skin of the particular area, one upper arm (deltoid) was not treated and served as control).

The healthy human skin is protected by a compact and regular layer of keratinized dead cells, filled by hydrophilic factors and surrounded by intercellular lipids. The cells and molecules of the stratum corneum, generated by metabolically active corneocytes of the inner layers, have a central role in the maintenance of skin miniaturization and hydration, working as a trap for water molecules.

When the cellular and biochemical organization of the stratum corneum is altered, the water content of the skin decreases.

In order to evaluate the hydrating power of cosmetics, the skin is topically treated with the test product for a period of 4 consecutive weeks, and instrumental evaluations of the stratum corneum water content are performed, before and after the cosmetic treatment.

The hydration of the outer layers of the skin was measured by instrumental evaluation of the water content in the stratum corneum (Corneomnetry).

Measurements were taken before (Tₒ) and after the test period (T_{f}), both on the test area and the control (not treated) area.

At the end of the test period (T_{f}) the final measurements were carried out about 4 hours after the last application.

The following variations were observed in the parameters taken into consideration, as compared to the baseline (Tₒ).

TABLE 17

| | T0 | Tf | p (*) | % variation |
|---|---|---|---|---|
| HYDRATANT SPF 15 (# 1) | 59.29 | 68.62 | <0.05 (*) | +15.74 |
| (Periocular Area) | | | | |
| NOT TREATED | 41.32 | 41.34 | >0.05 | +0.05 |
| (Upper arm - deltoid) | | | | |

(*) a value of p ≦ 0.05 is considered statistically significant

TABLE 18
PRODUCT HYDRATANT SPF 15 (# 1)
Tab. 1: Hydration levels before (T0) and after 4 weeks (Tf) of product
application in the treated area and in the control area (not treated).
All the hydration levels are expressed as corneometric units.

| | | | | Treated area | | Not Treated | |
|---|---|---|---|---|---|---|---|
| | | | | (Face - Periocular | | (Upper arm - | |
| | First | | | Area) | | deltoid) | |
| # Vol | Letter | Sex | Age | T0 | Tf | T0 | Tf |
| 1 | T. S. | F | 51 | 58.60 | 68.93 | 44.93 | 46.47 |
| 2 | M. M. | F | 49 | 54.80 | 63.63 | 41.87 | 41.87 |
| 3 | M. S. | F | 36 | 59.33 | 67.87 | 37.70 | 37.83 |
| 4 | B. L. | F | 30 | 66.13 | 78.60 | 43.50 | 43.20 |
| 5 | A. R. | F | 53 | 72.13 | 79.57 | 50.20 | 50.67 |
| 6 | M. L. | F | 45 | 67.57 | 74.50 | 42.13 | 42.63 |
| 7 | C. A. | F | 47 | 67.17 | 75.30 | 37.57 | 36.67 |
| 8 | P. L. | F | 46 | 64.87 | 73.43 | 36.10 | 37.63 |
| 9 | M. N. | F | 53 | 55.50 | 64.67 | 38.87 | 38.57 |
| 10 | R. L. | F | 50 | 61.80 | 71.63 | 40.13 | 40.20 |
| 11 | C. L. | F | 46 | 53.57 | 62.93 | 42.83 | 42.97 |
| 12 | D. P. | F | 66 | 58.97 | 69.03 | 36.90 | 36.40 |
| 13 | R. L. | F | 64 | 51.73 | 59.90 | 30.47 | 30.30 |
| 14 | C. N. | F | 42 | 60.20 | 71.00 | 48.50 | 48.83 |
| 15 | M. C. | F | 45 | 50.97 | 61.20 | 43.63 | 43.90 |
| 16 | E. A. | F | 53 | 56.70 | 67.80 | 44.30 | 44.30 |
| 17 | M. E. | F | 31 | 43.73 | 52.97 | 37.90 | 35.20 |
| 18 | M. L. | F | 62 | 54.30 | 64.30 | 44.97 | 44.37 |
| 19 | T. L. | F | 58 | 65.00 | 71.67 | 41.73 | 41.27 |
| 20 | V. P. | F | 58 | 60.77 | 73.40 | 40.23 | 40.03 |

It was further observed that the mean hydration level in the skin area treated with HYDRATANT SPF 15 (#1) was significantly improved as compared to the baseline value (A value of p≦0.05 is considered statistically significant), In the Not Treated control area the variation in the mean level of skin hydration was not statistically significant as compared to the baseline (p>0.05).

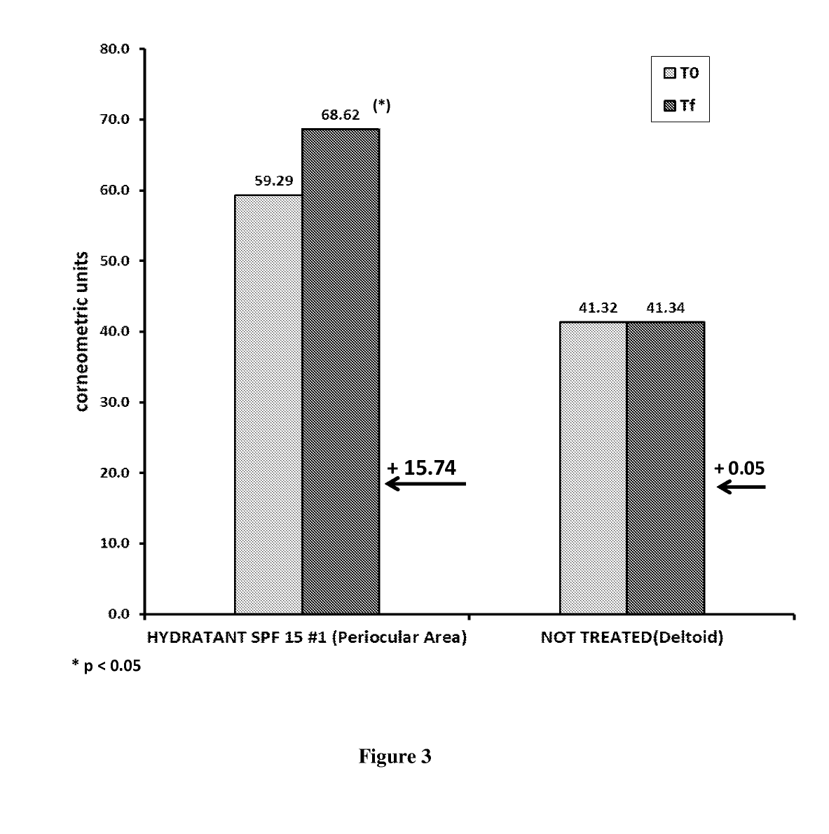

The mean hydration levels of treated control areas are illustrated graphically (FIG. 3). The data obtained are statistically analyzed and results are summarized in the tables 19, 20, 21 and graphical representations enclosed to the test results. Statistical evaluation of final measurements against baseline values is performed by two tail t-test for comparison of the means.

TABLE 19
Variations in the mean hydration levels before (T0) and after
4 weeks (Tf) of product application, expressed as
corneometric units.

| | | | | | % |
|---|---|---|---|---|---|
| Area | | T0 | Tf | p (*) | VARIATION |
| HYDRATANT | MEAN ± | 59.29 | 68.62 | <0.05 | +15.74 |
| SPF 15 (# 1) | S.D. | 6.94 | 6.64 | | |
| (Face - Periocular | | | | | |
| Area) | | | | | |
| NOT TREATED | MEAN ± | 41.32 | 41.34 | >0.05 | +0.05 |
| (Upper arm - deltoid) | S.D. | 4.44 | 4.68 | | |

In conclusion the cosmetic product HYDRATAMT SPF 15 (#1) appeared to be effective in significantly improving the hydration of the outer layers of the skin after 4 weeks of treatment.

### Example 6

### Sample #1

### Skin Profilometry

The evaluation of the three-dimensional skin profiles by software program primos 5.4 performed by a system of two-dimensional Fourier transformation.

Different skin surface parameters are determined by the PRIMOS software:

Ra=Arithmetic roughness Average

Rmax=Maximum roughness depth

Rt=Total Roughness: represents the arithmetic mean value of amplitude of the 5 highest profile peaks, and the 5 deepest profile valleys in the single measuring lengths

Rz=Mean roughness depth: represents the arithmetic mean of the roughness depth (Rt) measured in each of five segments of equal length in which each profile can be subdivided. This parameter gives the mean distance between deeper peaks and furrows and shows a good correlation with the sensorial roughness appearance of the skin surface

Rpm=Peak height average

Rvm=Valley depth mean

These parameters indicate different properties of the network of furrows.

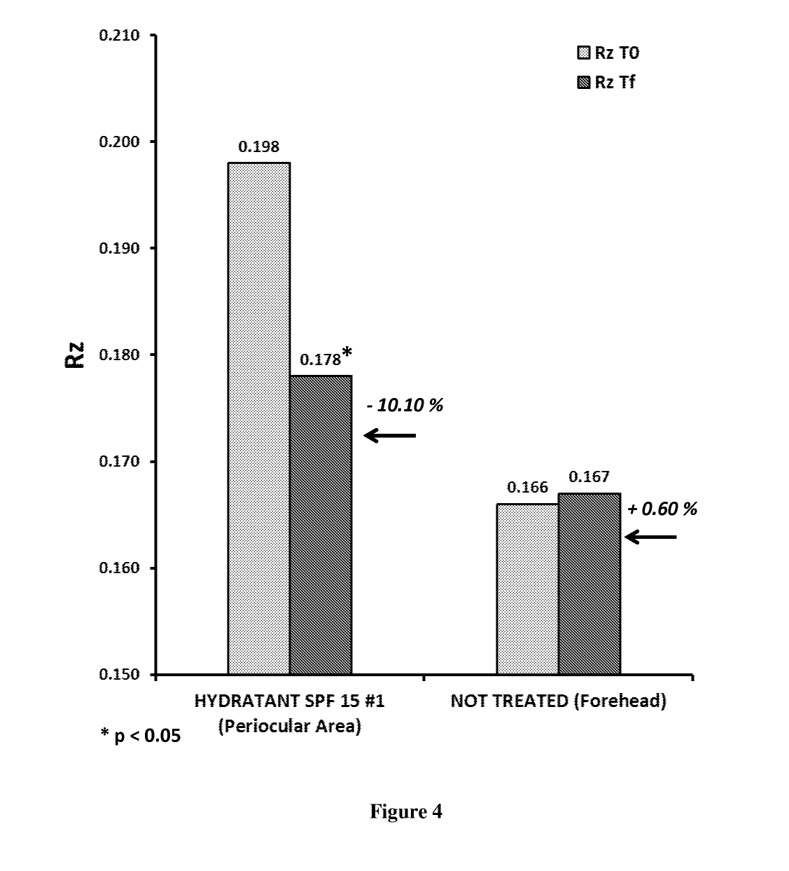

The parameter Rz is considered the most helpful to evaluate the efficacy of anti-wrinkle product. The data obtained are statistically analyzed and results are summarized in table 20, 21 and 22 below and graphical representations enclosed to the test results (see FIG. 4).

TABLE 20
The following variations were observed in the parameter Rz,
as compared to the baseline:

| AREA | Rz T0 | Rz Tf | p | % variation |
|---|---|---|---|---|
| Rz | 0.198 | 0.178 | <0.05 (*) | −10.10 |
| HYDRATANT SPF 15 (# 1) | | | | |
| Rz | 0.166 | 0.167 | >0.05 | +0.60 |
| Control Area | | | | |
| (Forehead) | | | | |

(*) a value of p ≦ 0.05 is considered statistically significant

TABLE 21
Rz: values before product application (T0) and after
4 consecutive weeks of product application (Tf) in the
treated area and in the control area (not treated).

| | | | | TREATED AREA | | Control area | |
|---|---|---|---|---|---|---|---|
| | First | | | (Face-Periocular area) | | (Forehead) | |
| # Vol | Letter | Sex | Age | T0 | Tf | T0 | Tf |
| 1 | T. S. | F | 51 | 0.213 | 0.192 | 0.220 | 0.219 |
| 2 | M. M. | F | 49 | 0.240 | 0.217 | 0.214 | 0.210 |
| 3 | M. S. | F | 36 | 0.151 | 0.125 | 0.163 | 0.169 |
| 4 | B. L. | F | 30 | 0.153 | 0.148 | 0.144 | 0.145 |
| 5 | A. R. | F | 53 | 0.223 | 0.215 | 0.112 | 0.117 |
| 6 | M. L. | F | 45 | 0.187 | 0.166 | 0.157 | 0.161 |
| 7 | C. A. | F | 47 | 0.158 | 0.135 | 0.158 | 0.157 |
| 8 | P. L. | F | 46 | 0.198 | 0.189 | 0.143 | 0.148 |
| 9 | M. N. | F | 53 | 0.167 | 0.153 | 0.150 | 0.156 |
| 10 | R. L. | F | 50 | 0.256 | 0.248 | 0.127 | 0.131 |
| 11 | C. L. | F | 46 | 0.192 | 0.159 | 0.153 | 0.148 |
| 12 | D. P. | F | 66 | 0.216 | 0.202 | 0.231 | 0.238 |
| 13 | R. L. | F | 64 | 0.286 | 0.215 | 0.161 | 0.160 |
| 14 | C. N. | F | 42 | 0.205 | 0.171 | 0.182 | 0.184 |
| 15 | M. C. | F | 45 | 0.273 | 0.252 | 0.140 | 0.143 |
| 16 | E. A. | F | 53 | 0.165 | 0.160 | 0.168 | 0.163 |
| 17 | M. E. | F | 31 | 0.152 | 0.133 | 0.121 | 0.117 |
| 18 | M. L. | F | 62 | 0.154 | 0.127 | 0.212 | 0.206 |
| 19 | T. L. | F | 58 | 0.208 | 0.202 | 0.145 | 0.147 |
| 20 | V. P. | F | 58 | 0.165 | 0.156 | 0.219 | 0.219 |

TABLE 22
Rz: Variations in the mean Rz levels before (T0) and after
4 consecutive weeks (Tf) of treatment.

| | | | | | % |
|---|---|---|---|---|---|
| | | T0 | Tf | p | variation |
| Rz | MEAN ± | 0.198 | 0.178 | <0.05 (*) | −10.10 |
| HYDRATANT | S.D. | 0.042 | 0.039 | | |
| SPF 15 (# 1) | | | | | |
| (Periocular area) | | | | | |
| Rz | MEAN ± | 0.166 | 0.167 | >0.05 | +0.60 |
| Control Area | S.D. | 0.035 | 0.036 | | |
| (Forehead) | | | | | |

(*) a value of p ≦ 0.05 is considered statistically significant

The potential efficacy of the cosmetic product HYDRETANT SPF 15 (#1) to improve the appearance of skin micro relief and to attenuate the appearance of fine lines and wrinkles was evaluated on a panel constituted of 20 volunteers ‘female, aged 20-70 years (two applications per day, morning and evening ‘on the skin of the face ‘including periocular area; a skin area on forehead was not treated and served as control. 3D skin profiles were taken before (To) and after 4 consecutive weeks of use (T_{f}), both on treated and control (not treated) areas.

The parameter Rz was taken into consideration, in order to evaluate changes in the skin surface: Rz (Mean roughness depth) represents the arithmetic mean of the roughness depth (Rₜ) measured in each of five segments of equal length in which each profile can be subdivided. This parameter gives the mean distance between deeper peaks and furrows and shows a good correlation with the sensorial roughness appearance of the skin surface.

The observed decrease of the parameter above reported indicates a global and statistically significant improvement in the appearance of skin surface, as well as the diminution of the fine lines and wrinkles in the treated area, while no significant variation has been observed in the control area at the end of the test period.

It can therefore be concluded that cosmetic product HYDRETANT SPF 15 (sample #1), in the adopted test conditions, appearance to be active in improving the softness of the skin and attenuating the appearance of fine lines and wrinkles, hence improving the global appearance of the skin microrelief, as confirmed by statistical analysis.

### Example 7

### Sample #2

### Improvement of Elastic Properties of Human Skin

HYDRATANT SPF 15 sample #2, was prepared and tested according to the same principle and method presented in Example 4.

The potential efficacy of the cosmetic product HYDRATANT SPF 15 (sample #2) to improve the elastic properties of the skin was evaluated on a period constituted of 20 volunteers, female, aged 20-70 years (two applications per day, morning and evening), on the skin of one inner forearm, a skin area chosen on the upper arm. (deltoid) was not treated and served as control.

The parameters describing the elastic properties of the outer layers of the skin were evaluated by instrumental measurements (Cutometer MPS 580 Courage & Khazaka). Measurements were taken before (Tₒ) and after the test period (T_{f}), both on the test area and the control (not treated) area.

At the end of the test period (T_{f}) the final measurements were carried out about 4 hours after the last product application.

The following variations were observed in the parameters taken into consideration, as compared to the baseline (Tₒ).

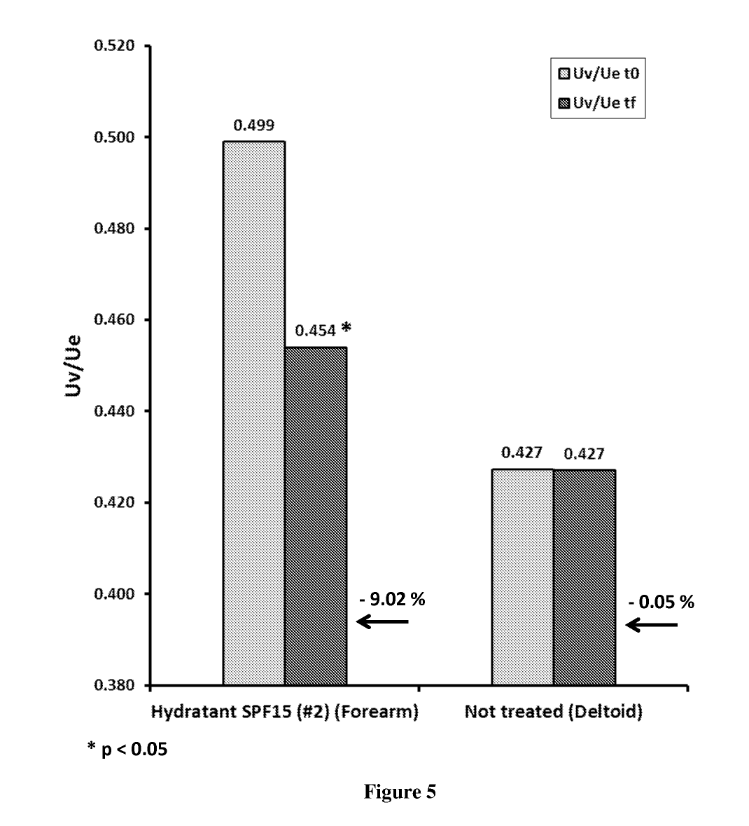

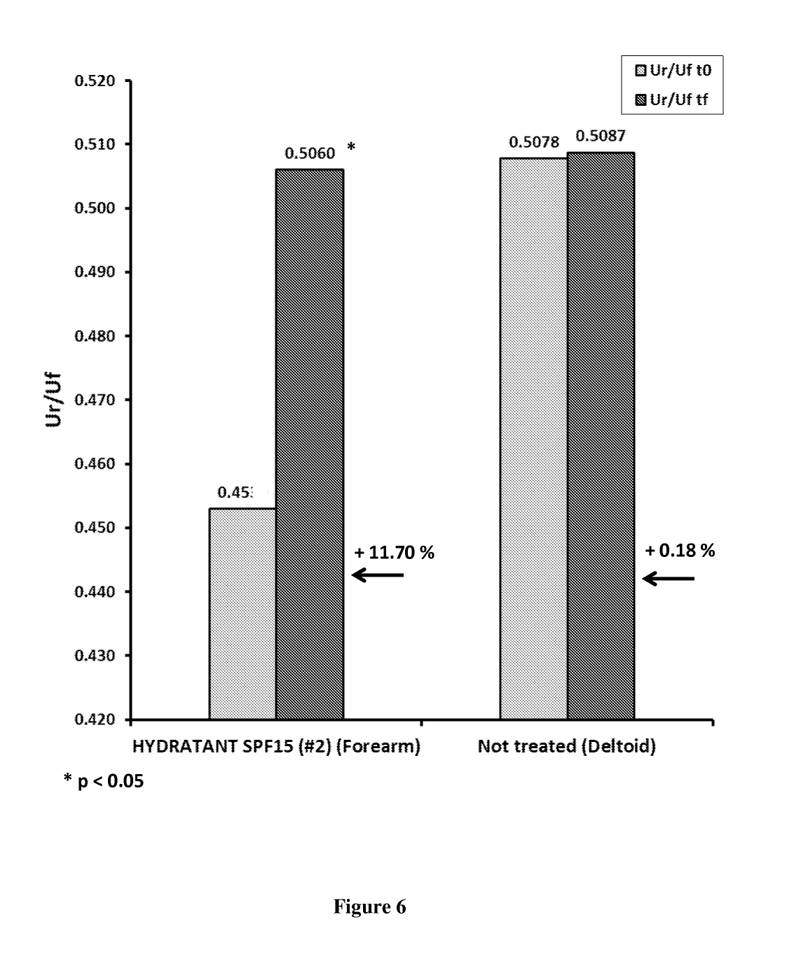

The data obtained are statistically analyzed and results are summarized in table 23 and 24 below and graphical representations enclosed to the test results (see FIG. 5 and FIG. 6).

TABLE 23
PRODUCT: HYDRATANT SPF 15 (# 2)
TREATED AREA - Uv/Ue and Ur/Uf: values before product application
(T0) and after 4 consecutive weeks of product application (Tf).

| | First | | | Uv/Ue | Uv/Ue | Ur/Uf | Ur/Uf |
|---|---|---|---|---|---|---|---|
| # Vol | letter | Sex | AGE | T₀ | T_{f} | T₀ | T_{f} |
| 1 | T. S. | F | 51 | 0.271 | 0.242 | 0.461 | 0.534 |
| 2 | M. M. | F | 49 | 0.498 | 0.457 | 0.377 | 0.433 |
| 3 | M. S. | F | 36 | 0.513 | 0.485 | 0.517 | 0.610 |
| 4 | B. L. | F | 30 | 0.436 | 0.416 | 0.546 | 0.544 |
| 5 | A. R. | F | 53 | 0.689 | 0.634 | 0.415 | 0.483 |
| 6 | M. L. | F | 45 | 0.449 | 0.412 | 0.470 | 0.513 |
| 7 | C. A. | F | 47 | 0.796 | 0.741 | 0.506 | 0.571 |
| 8 | P. L. | F | 46 | 0.295 | 0.264 | 0.533 | 0.600 |
| 9 | M. N. | F | 53 | 0.413 | 0.390 | 0.354 | 0.423 |
| 10 | R. L. | F | 50 | 0.621 | 0.562 | 0.416 | 0.452 |
| 11 | C. L. | F | 46 | 0.508 | 0.434 | 0.475 | 0.545 |
| 12 | D. P. | F | 66 | 0.555 | 0.511 | 0.320 | 0.359 |
| 13 | R. L. | F | 64 | 0.298 | 0.248 | 0.409 | 0.464 |
| 14 | C. N. | F | 42 | 0.551 | 0.496 | 0.478 | 0.523 |
| 15 | M. C. | F | 45 | 0.360 | 0.294 | 0.505 | 0.527 |
| 16 | E. A. | F | 53 | 0.521 | 0.472 | 0.403 | 0.462 |
| 17 | M. E. | F | 31 | 0.576 | 0.531 | 0.542 | 0.603 |
| 18 | M. L. | F | 62 | 0.551 | 0.506 | 0.450 | 0.532 |
| 19 | T. L. | F | 58 | 0.487 | 0.450 | 0.422 | 0.474 |
| 20 | V. P. | F | 58 | 0.593 | 0.544 | 0.468 | 0.470 |

TABLE 24
CONTROL AREA (Not Treated) - Uv/Ue and Ur/Uf: values before (T0)
and after 4 consecutive weeks of product application (Tf).

| | First | | | Uv/Ue | Uv/Ue | Ur/Uf | Ur/Uf |
|---|---|---|---|---|---|---|---|
| # Vol | letter | Sex | AGE | T₀ | T_{f} | T₀ | T_{f} |
| 1 | T. S. | F | 51 | 0.251 | 0.258 | 0.574 | 0.599 |
| 2 | M. M. | F | 49 | 0.491 | 0.502 | 0.514 | 0.526 |
| 3 | M. S. | F | 36 | 0.346 | 0.331 | 0.478 | 0.478 |
| 4 | B. L. | F | 30 | 0.455 | 0.458 | 0.576 | 0.562 |
| 5 | A. R. | F | 53 | 0.549 | 0.544 | 0.553 | 0.513 |
| 6 | M. L. | F | 45 | 0.462 | 0.460 | 0.467 | 0.480 |
| 7 | C. A. | F | 47 | 0.593 | 0.593 | 0.452 | 0.445 |
| 8 | P. L. | F | 46 | 0.282 | 0.289 | 0.547 | 0.595 |
| 9 | M. N. | F | 53 | 0.332 | 0.325 | 0.518 | 0.519 |
| 10 | R. L. | F | 50 | 0.585 | 0.579 | 0.470 | 0.480 |
| 11 | C. L. | F | 46 | 0.439 | 0.439 | 0.591 | 0.599 |
| 12 | D. P. | F | 66 | 0.479 | 0.478 | 0.388 | 0.372 |
| 13 | R. L. | F | 64 | 0.524 | 0.526 | 0.480 | 0.482 |
| 14 | C. N. | F | 42 | 0.225 | 0.230 | 0.534 | 0.532 |
| 15 | M. C. | F | 45 | 0.237 | 0.237 | 0.621 | 0.594 |
| 16 | E. A. | F | 53 | 0.301 | 0.303 | 0.512 | 0.516 |
| 17 | M. E. | F | 31 | 0.553 | 0.553 | 0.481 | 0.485 |
| 18 | M. L. | F | 62 | 0.429 | 0.412 | 0.349 | 0.342 |
| 19 | T. L. | F | 58 | 0.459 | 0.452 | 0.512 | 0.515 |
| 20 | V. P. | F | 58 | 0.554 | 0.574 | 0.539 | 0.541 |

The following variations were observed in the parameters taken into consideration, as compared to the baseline (Tₒ): Viscoelastic to elastic ratio (Uv/Ue): at the end of the application period the mean value of the parameter showed a decrease of 9.02% (p<0.05) in the treated area, and a decrease of 0.05% in the control area (p>0.05).

Decreasing values of Uv/Ue indicate a decreasing viscoelastic portion of the deformation process and a relative increase of the elastic portion (a decrease of this parameter show a general improvement of skin elastic properties).

Biologic elasticity (Ur/Uf): at the end of the application period, the mean value of this parameter showed an increase of 11.70% (p<0.05) in the treated area, and an increase of 0.18% in the control area (p>0.05).

This parameter shows the ability of the skin to regain its initial position after deformation (an increase of this parameter is indicative of a general improvement of skin classic properties).

The variation observed in these two parameters show a global improvement in the elastic properties of the outer layers of the skin, following the application of the cosmetic product HYDRATANT SPF 15 (#2).

In the control area (not treated) the variation in the skin elastic properties were not statistically significant as compared to the baseline (p>0.05).

TABLE 25
Uv/Ue and Ur/Uf variations in the mean parameters levels describing
the elastic properties of the skin before (T0) and after 4 consecutive
weeks of treatment (Tf)

| | | | | | % |
|---|---|---|---|---|---|
| | | t₀ | t_{f} | P | variation |
| Uv/Ue | MEAN ± | 0.499 | 0.454 | <0.05 (*) | −9.02 |
| HYDRATANT SPF 15 (# 2) | S.D. | 0.132 | 0.27 | | |
| Uv/Ue | MEAN ± | 0.4273 | 0.4271 | >0.05 | −0.05 |
| CONTROL AREA | S.D. | 0.121 | 0.121 | | |
| (not treated) | | | | | |
| Ur/Uf | MEAN ± | 0.453 | 0.506 | <0.05 (*) | +11.70 |
| HYDRATANT SPF 15 (# 2) | S.D. | 0.0636 | 0.066 | | |
| Ur/Uf | MEAN ± | 0.5078 ± 0.066 | 0.5087 ± 0.069 | >0.05 | +0.18 |
| CONTROL AREA | S.D. | | | | |
| (not treated) | | | | | |

(*) a value of p ≦ 0.05 is considered statistically significant

The following variations were observed in the parameters taken into consideration, as compared to the baseline (Tₒ):

Viscoelastic to elastic ratio (Uv/Ue): at the end of the application period the mean value of the parameter showed a decrease of 9.02% (p<0.05) in the treated area, and a decrease of 0.05% in the control area (p>0.05).

Decreasing values of Uv/Ue indicate a decreasing viscoelastic portion of the deformation process and a relative increase of the elastic portion (a decrease of this parameter show a general improvement of skin elastic properties).

Biologic elasticity (Ur/Uf): at the end of the application period, the mean value of this parameter showed an increase of 11.70% (p<0.05) in the treated area, and an increase of 0.18% in the control area (p>0.05).

This parameter shows the ability of the skin to regain its initial position after deformation (an increase of this parameter is indicative of a general improvement of skin classic properties).

The variation observed in these two parameters show a global improvement in the elastic properties of the outer layers of the skin, following the application of the cosmetic product HYDRATANT SPF 15 (sample #2).

In the control area (not treated) the variation in the skin elastic properties were not statistically significant as compared to the baseline (p>0.05).

The viscoelastic to elastic rate (Uv/Ue) increasing values of Uv/Ue indicate an increasing viscoelastic portion of the deformation process and a relative decrease of the elastic portion. A decrease of this parameter reflects a general improvement in the elastic properties of the skin.

Biologic elasticity (Ur/Uf) it shows the ability of the skin to regain its initial position after deformation. A value of 1 would increase 100% elasticity. An increase of the parameter reflects a general improvements in the elastic properties of the skin.

It can therefore be concluded that the cosmetic product HYDRATANT SPF 15 (sample #2) applied in the adopted test conditions, appears to be active in improving the elastic properties of the outer layers of the skin: skin deformability appeared significantly reduced and skin elasticity appeared significantly increased after 4 conservative weeks of product application.

In the control area (not treated) the variation in the skin elastic properties were not statistically significant as compared to the baseline (p>0.05).

### Example 8

### Sample #2 Hydrating Activity

HYDRATANT SPF 15 sample #2, was prepared and tested according to the same principle and method presented in Example 5.

The hydrating activity of the cosmetic product HYDRATANT SPF 15 (#2) was evaluated after a period of 4 consecutive weeks of application on a panel constituted of 20 volunteers, female, aged 20-70 (two applications per day. Morning and evening, on the skin of the particular area, one upper arm (deltoid) was not treated and served as control).

We measured the hydration of the outer layers of the skin by instrumental evaluation of the water content in the stratum corneum (Corneomnetry).

Measurements were taken before (Tₒ) and after the test period (T_{f}), both on the test area and the control (not treated) area.

At the end of the test period (Tf) the final measurements were carried out about 4 hours after the last application.

The following variations were observed in the parameters taken into consideration, as compared to the baseline (Tₒ).

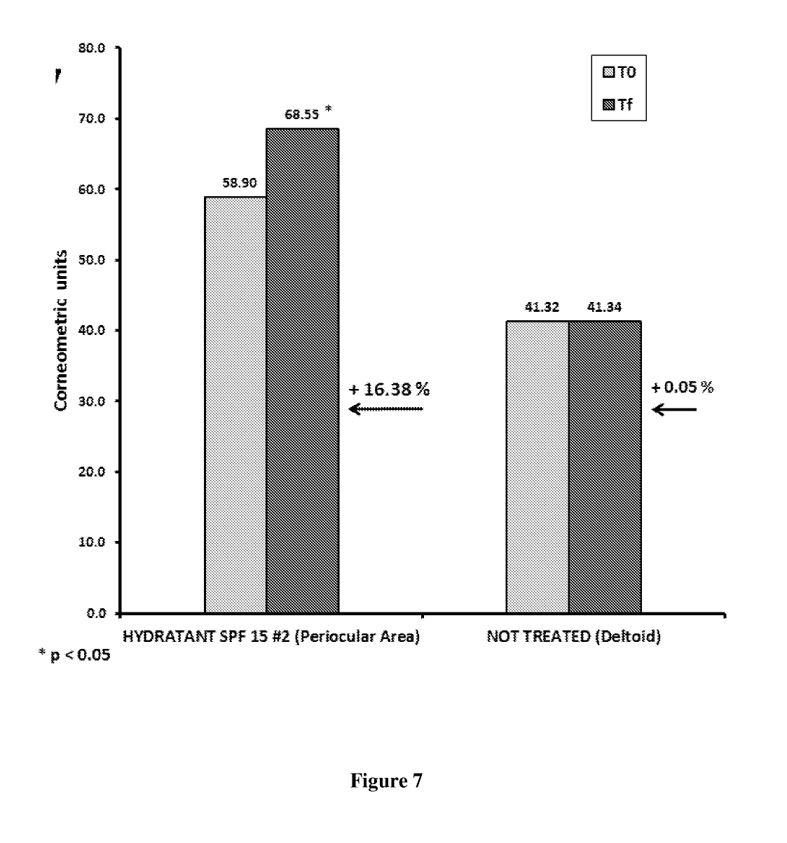

The data obtained are statistically analyzed and results are summarized in table 26, 27 and 28) below and graphical representations enclosed to the test results (see FIG. 7 and FIG. 2).

TABLE 26
The following variations were observed in the parameters taken
into consideration, as compared to the baseline (T0):

| | T0 | Tf | p (*) | % variation |
|---|---|---|---|---|
| HYDRATANT SPF 15 (# 2) | 58.90 | 68.55 | <0.05 (*) | +16.38 |
| (Periocular Area) | | | | |
| NOT TREATED | 41.32 | 41.34 | >0.05 | +0.05 |
| (Upper arm - deltoid) | | | | |

(*) a value of p ≦ 0.05 is considered statistically significant

TABLE 27
Variations in the mean hydration levels before (T0) and after
4 weeks (Tf) of product application, expressed as
corneometric units.

| | | | | | % |
|---|---|---|---|---|---|
| Area | | T0 | Tf | p (*) | VARIATION |
| HYDRATANT | MEAN ± | 58.90 | 68.55 | <0.05 (*) | +16.38 |
| SPF 15 (# 2) | S.D. | 7.18 | 7.25 | | |
| (Face - Periocular | | | | | |
| Area) | | | | | |
| NOT TREATED | MEAN ± | 41.32 | 41.34 | >0.05 | +0.05 |
| (Upper arm - | S.D. | 4.44 | 4.68 | | |
| Deltoid) | | | | | |

(*) a value of p ≦ 0.05 is considered statistically significant

At the end of the test we observed that the mean hydration level in the skin area treated with HYDRATANT SPF 15 (#2) was significantly improved as compared to the baseline value (p<0.05).

In the Not Treated control area the variation in the mean level of skin hydration was not statistically significant as compared to the baseline (p>0.05).

In conclusion the cosmetic product HYDRATAMT SPF 15 (#2) appeared to be effective in significantly improving the hydration of the outer layers of the skin after 4 weeks of treatment.

### Example 9

### Sample #2

### Skin Profilometry

HYDRATANT SPF 15 sample #2, was prepared and tested according to the same principle and method presented in Example 6.

The evaluation of the three-dimensional skin profiles by software program primos 5.4 performed by a system of two-dimensional Fourier transformation.

Different skin surface parameters are determined by the PRIMOS software:

Ra=Arithmetic roughness Average

Rmax=Maximum roughness depth

Rt=Total Roughness: represents the arithmetic mean value of amplitude of the 5 highest profile peaks, and the 5 deepest profile valleys in the single measuring lengths

Rz=Mean roughness depth: represents the arithmetic mean of the roughness depth (Rt) measured in each of five segments of equal length in which each profile can be subdivided. This parameter gives the mean distance between deeper peaks and furrows and shows a good correlation with the sensorial roughness appearance of the skin surface

Rpm=Peak height average

Rvm=Valley depth mean

These parameters indicate different properties of the network of furrows.

The parameter Rz is considered the most helpful to evaluate the efficacy of anti-wrinkle product.

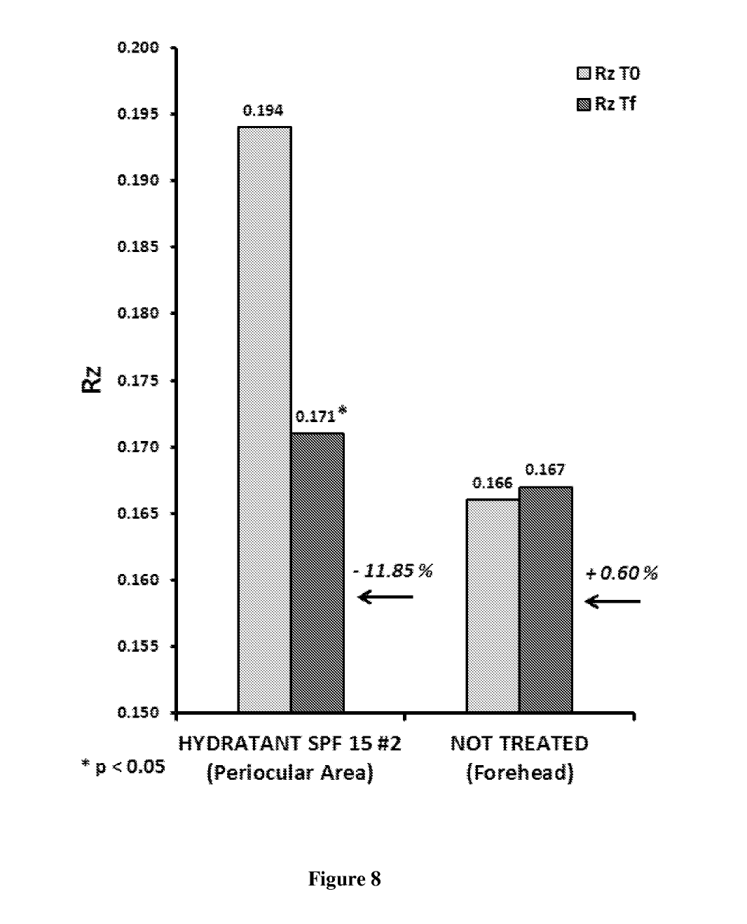

The data obtained are statistically analyzed and results are summarized in Tables 28, 29 and 30 below and graphical representations enclosed to the test results (see FIG. 8).

TABLE 28
The following variations were observed in the parameter Rz,
as compared to the baseline:

| AREA | Rz T0 | Rz Tf | p | % variation |
|---|---|---|---|---|
| Rz | 0.194 | 0.171 | <0.05 (*) | −11.85 |
| HYDRATANT SPF 15 (# 2) | | | | |
| Rz | 0.166 | 0.167 | >0.05 | +0.60 |
| Control Area | | | | |
| (Forehead) | | | | |

(*) a value of p ≦ 0.05 is considered statistically significant

As it appears from the table over reported, the parameter Rz is significantly improved in the skin area treated with the product HYDRATANT SPF 15 (#2) (a decrease in the parameter Rz indicates an improvement in attenuating the appearance of fine lines and wrinkles) as compared to the baseline, after 4 continuative weeks of application,

In the control area (not treated) the variations in the mean levels of the parameter Rz are not statistically significant as compared to the baseline after 4 continuative weeks of test period.

TABLE 29
PRODUCT: HYDRATANT SPF 15 (# 2)
Rz: values before product application (T0) and after
4 consecutive weeks of product application (Tf) in the
treated area and in the control area (not treated).

| | | | | TREATED AREA | | Control area | |
|---|---|---|---|---|---|---|---|
| | First | | | (Face-Periocular area) | | (Forehead) | |
| # Vol | Letter | Sex | Age | T0 | Tf | T0 | Tf |
| 1 | T. S. | F | 51 | 0.227 | 0.199 | 0.220 | 0.219 |
| 2 | M. M. | F | 49 | 0.265 | 0.238 | 0.214 | 0.210 |
| 3 | M. S. | F | 36 | 0.138 | 0.111 | 0.163 | 0.169 |
| 4 | B. L. | F | 30 | 0.156 | 0.148 | 0.144 | 0.145 |
| 5 | A. R. | F | 53 | 0.262 | 0.242 | 0.112 | 0.117 |
| 6 | M. L. | F | 45 | 0.194 | 0.165 | 0.157 | 0.161 |
| 7 | C. A. | F | 47 | 0.155 | 0.140 | 0.158 | 0.157 |
| 8 | P. L. | F | 46 | 0.128 | 0.126 | 0.143 | 0.148 |
| 9 | M. N. | F | 53 | 0.186 | 0.160 | 0.150 | 0.156 |
| 10 | R. L. | F | 50 | 0.244 | 0.200 | 0.127 | 0.131 |
| 11 | C. L. | F | 46 | 0.159 | 0.148 | 0.153 | 0.148 |
| 12 | D. P. | F | 66 | 0.277 | 0.263 | 0.231 | 0.238 |
| 13 | R. L. | F | 64 | 0.188 | 0.147 | 0.161 | 0.160 |
| 14 | C. N. | F | 42 | 0.189 | 0.155 | 0.182 | 0.184 |
| 15 | M. C. | F | 45 | 0.273 | 0.248 | 0.140 | 0.143 |
| 16 | E. A. | F | 53 | 0.169 | 0.158 | 0.168 | 0.163 |
| 17 | M. E. | F | 31 | 0.146 | 0.121 | 0.121 | 0.117 |
| 18 | M. L. | F | 62 | 0.177 | 0.148 | 0.212 | 0.206 |
| 19 | T. L. | F | 58 | 0.182 | 0.158 | 0.145 | 0.147 |
| 20 | V. P. | F | 58 | 0.165 | 0.156 | 0.219 | 0.219 |

TABLE 30
PRODUCT: HYDRATANT SPF 15 (# 2)
Rz: Variations in the mean Rz levels before (T0) and after
4 consecutive weeks (Tf) of treatment.

| | | | | | % |
|---|---|---|---|---|---|
| | | T0 | Tf | p | variation |
| Rz | MEAN ± | 0.194 | 0.171 | <0.05 (*) | −11.85 |
| HYDRATANT | S.D. | 0.047 | 0.045 | | |
| SPF 15 (# 2) | | | | | |
| (Periocular area) | | | | | |
| Rz | MEAN ± | 0.166 | 0.167 | >0.05 | +0.60 |
| Control Area | S.D. | 0.035 | 0.036 | | |
| (Forehead) | | | | | |

(*) a value of p ≦ 0.05 is considered statistically significant

The potential efficacy of the cosmetic product HYDRETANT SPF 15 (#2) to improve the appearance of skin microrelief and to attenuate the appearance of fine lines and wrinkles was evaluated on a panel constituted of 20 volunteers ‘female, aged 20-70 years (two applications per day, morning and evening ‘on the skin of the face ‘including periocular area; a skin area on forehead was not treated and served as control 3D skin profiles were taken before (To) and after 4 consecutive weeks of use (T_{f}), both on treated and control (not treated) areas.

The parameter Rz was taken into consideration, in order to evaluate changes in the skin surface: Rz (Mean roughness depth) represents the arithmetic mean of the roughness depth (Rt) measured in each of five segments of equal length in which each profile can be subdivided. This parameter gives the mean distance between deeper peaks and furrows and shows a good correlation with the sensorial roughness appearance of the skin surface.

The observed decrease of the parameter above reported indicates a global and statistically significant improvement in the appearance of skin surface, as well as the diminution of the fine lines and wrinkles in the treated area, while no significant variation has been observed in the control area at the end of the test period.

Table 31 below summarizes sample #1 and sample #2 results and efficacy:

| | Not treated area | | |
|---|---|---|---|
| | (Control) | sample# 1 | sample # 2 |
| | | Treated area | Treated area |
| SKIN HYDRATION | Upper Arm - Deltoid | (Face - periocular area) | (Face - periocular area) |
| Hydration of the outer layers of the skin by instrumental evaluation | +0.05 | +15.74% (*) | +16.38% (*) |
| of the water content in the stratum corneum | | | |
| An INCREASE of this parameter shows a general improvement | | | |
| of skin hydration | | | |
| | Not treated area | | |
| | (Control) | sample # 1 | sample # 2 |
| | | Treated area | Treated area |
| SKIN PROFILOMETRY | Forehead | (Face - periocular area) | (Face - periocular area) |
| A DECREASE in the parameter indicates an improvement in the | +0.60% | −10.10% (*) | −11.85% (*) |
| appearance of fine lines and wrinkles | | | |
| | Not treated area | | |
| | (Control) | sample # 1 | sample # 2 |
| SKIN ELASTOMETRY | Upper Arm - Deltoid | Treated area (Forearm) | Treated area (Forearm) |
| Two relative parameters indicative of mechanical properties of the skin | −0.05% | −8.60% (*) | −9.02% (*) |
| are calculated: | | | |
| 1) The viscoelastic to elastic ratio (Uv/Ue): Decreasing values of Uv/Ue | | | |
| indicates a decreasing viscoelastic portion of the deformation process | | | |
| and a relative increase of the elastic portion. A decrease of this parameter | | | |
| shows a general improvement of skin elastic properties. | | | |
| 2) Biologic elasticity (Ur/Uf): it shows the ability of the skin to regain | +0.18% | +9.76% (*) | +11.70% (*) |
| its initial position after deformation. A theoretical value of 1 would | | | |
| indicate 100% elasticity. An INCREASE of this parameter shows | | | |
| a general improvement of skin elastic properties | | | |

(*) a value of p ≦ 0.05 is considered statistically significant. All the variations marked with * are statistically significant as compared to baseline.

It can therefore be concluded that cosmetic product HYDRETANT SPF 15 (#1 and #2), in the adopted test conditions, appearance to be active in improving the skin elasticity, skin hydration, softness of the skin and attenuating the appearance of fine lines and wrinkles, hence improving the global appearance of the skin microrelief, as confirmed by statistical analysis.

1-82. (canceled)

83. A topical composition for providing a skin improvement, said composition characterized by at least one type of irradiated crystals and an aqueous preparation, said crystal irradiated according to at least one frequencies scheme selected from the group consisting of a range of between about 0.1 and 1000 Hz, about 0.2 and 200 Hz, a range of between about 1 and 150 Hz and any combination thereof; said improvement is selected from the group consisting of increasing skin biologic elasticity, decreasing skin viscoelastic, increasing skin hydration, decreasing of skin wrinkles and skin fine lines, skin regeneration and any combination thereof.

84. The topical composition according to claim 83, wherein said crystal are irradiated according to a predetermined intensities range selected from the group consisting of a range of 1 and 1000 μT, a range of 10 and 500 μT and any combination thereof.

85. The topical composition according to claim 83, wherein at least one of the following holds true:
a. said frequencies scheme includes a first sequence of said frequencies increasing linearly and continuously from a first frequency to a second, greater, frequency, and a second sequence of said frequencies decreasing linearly and continuously from said second frequency to said first frequency;
b. said composition when applied topically associates with an increase of more than 9% in skin biologic elasticity;
c. said composition when applied topically correlates with a decrease of at least 8% in skin viscoelastic portion of the deformation process and an increase of the elastic properties, respectively;
d. said composition when applied topically increase of more than 15% of skin hydration; and
e. said composition when applied topically provides a decrease of at least 10% of skin profilometry, thereby improvement of wrinkle and fine line appearance of said skin.

86. The topical composition according to claim 83, wherein at least one of the following holds true:
a. said crystal is made of sucrose, silicon, pearl, smart pearl, quartz, smart patches, smart particles, encoded gems, encoded stones, encoded food supplements or any other inert material and a combination thereof;
b. wherein said aqueous preparation is in the form of a lotion, mixture, compound, formulation, gel, cream, serum, liquid infusion, spray, suspension, emulsion, emollient preparation, emollient moisturizer and any combination thereof; and
c. said skin improvements further includes parameters and/or disorders selected from the group consisting of trophism of the integumentary system, regeneration of skin cells, skin irritation, skin infection, skin inflammation, the effectiveness of the skin as a barrier, skin aging, skin pigmentation, allergy, dermatitis, allergic dermatitis, skin degeneration or atrophy and any combination thereof;

87. A method of providing a skin improvement selected from the group consisting of increasing skin biologic elasticity, decreasing skin viscoelastic, increasing skin hydration, decreasing of skin wrinkles and skin fine lines, skin regeneration and any combination thereof; said method comprising steps of:
a. applying a composition comprising at least one type of irradiated crystals and an aqueous preparation; said irradiated crystals are exposed to a frequencies scheme selected from the group consisting of a range of between about 0.1 and 1000 Hz, about 0.2 and 200 Hz, a range of between about 1 and 150 Hz and any combination thereof.

88. The method according to claim 87, wherein said method additionally comprising step of exposing said irradiated crystals to a predetermined intensities range selected from the group consisting of a range of 1 and 1000 μT, a range of 10 and 500 μT and any combination thereof.

89. The method according to claim 87, wherein said method additionally comprising step of providing said irradiated crystals exposed to frequencies scheme including a first sequence of said frequencies increasing linearly and continuously from a first frequency to a second, greater, frequency, and a second sequence of said frequencies decreasing linearly and continuously from said second frequency to said first frequency.

90. A composition comprising at least one type of irradiated crystals and an aqueous preparation; wherein said irradiated crystals are exposed to a changing electromagnetic field.

91. The composition according to claim 90, wherein at least one of the following holds true:
a. said changing electromagnetic field is applied according to a predetermined protocol;
b. said changing electromagnetic field oscillates at a frequency in the range of between about 0.1 and 1000 Hz;
c. said changing electromagnetic field oscillates at a frequency in the range of between about 0.2 and 200 Hz;
d. said changing electromagnetic field oscillates at a frequency in the range of between about 1 and 150 Hz; and
e. said changing electromagnetic field is applied in increased frequencies.

92. The composition according to claim 90, wherein at least one of the following holds true:
a. said changing electromagnetic field is applied in a series of predetermined intensities in the range of 1 and 1000 μT;
b. said changing electromagnetic field is applied in a series of predetermined intensities in the range of 10 and 500 μT, preferably 5 to 200 μT;
c. said changing electromagnetic field is applied in a continuous waveform;
d. said changing electromagnetic field is applied in a pulsed waveform; and
e. said crystal is made of sucrose, silicon, pearl, smart pearls, quartz, smart patches, smart particles, encoded gems, encoded stones, encoded food supplements or any other inert material and a combination thereof.

93. The composition according to claim 90, wherein at least one of the following holds true:
a. said aqueous preparation is in the form of a lotion, mixture, compound, formulation, gel, cream, serum, liquid infusion, spray, suspension, emulsion, emollient preparation, emollient moisturiser and any combination thereof;
b. said composition is adapted to improve or treat at least one medical parameter or condition in a mammal, preferably in a human;
c. said composition is adapted to prevent at least one medical condition in a mammal, preferably in a human;
d. said composition is adapted to stimulate the regeneration and/or the recovery of an organ, tissue or a system or a portion thereof in a mammal, preferably in a human; and
e. said medical parameter or condition is selected from the group consisting of skin related parameters and/or disorders, recovery of an organ, tissue or system or a portion thereof, brain related parameters and any combination thereof.

94. The composition according to claim 90, wherein at least one of the following holds true:
a. said skin related parameters and/or disorders are selected from the group consisting of trophism of the integumentary system, regeneration of skin cells, skin irritation, skin infection, skin inflammation, the effectiveness of the skin as a barrier, skin aging, skin pigmentation, allergy, dermatitis, allergic dermatitis, skin degeneration or atrophy and any combination thereof;
b. said organ, tissue or system is selected from the group consisting of the lymph system, the endocrine system, the liver, skin, the integumentary system and any portion or combination thereof;
c. said brain related parameters are selected from the group consisting of regulation and/or controlling of the muscle tonus center and/or motor control center of the cerebellum, the reflex centers, regulation or controlling of the posterior pituitary (Neurohypophysis) of the pituitary gland or hypophysis, regulation of the skin center in the brain and any combination thereof; and
d. said composition is applied topically to the integuments and covering or support tissues of mammals, in human or veterinary fields.

95. A process for the preparation of a composition for prevention, treatment, stimulation and/or recovery of at least a portion of a mammalian tissue or system, preferably the skin system, comprising the steps of:
a. providing an aqueous preparation;
b. providing at least one type of crystal;
c. exposing said at least one type of crystal to a changing electromagnetic field so as to obtain at least one type of an irradiated crystals;
d. contacting said at least one type of irradiated crystals with said aqueous preparation thereby producing said composition for prevention, treating, and/or stimulating the recovery of said at least a portion of a mammalian tissue or system, preferably the skin system.

96. The process according to claim 95, wherein at least one of the following holds true:
a. said process comprising an additional step of exposing said at least one type of crystal to a changing electromagnetic field according to a predetermined protocol;
b. said process comprising an additional step of exposing said at least one type of crystal to a changing electromagnetic field oscillating at a frequency in the range of between about 0.1 and 1000 Hz;
c. said process comprising an additional step of exposing said at least one type of crystal to a changing electromagnetic field oscillating at a frequency in the range of between about 0.2 and 200 Hz;
d. said process comprising an additional step of exposing said at least one type of crystal to a changing electromagnetic field oscillating at a frequency in the range of between about 1 and 150 Hz; and
e. said process comprising an additional step of exposing said at least one type of crystal to a changing electromagnetic field applied in increased frequencies.

97. The process according to claim 95, wherein at least one of the following holds true:
a. said step of exposing said at least one type of crystal to a changing electromagnetic field is applied in a series of predetermined intensities in the range of 1 and 1000 μT;
b. said step of exposing said at least one type of crystal to a changing electromagnetic field is applied in a series of predetermined intensities in the range of 10 and 500 μT, preferably 5 to 200 μT;
c. said step of exposing said at least one type of crystal to a changing electromagnetic field is applied in a continuous waveform; and
d. said step of exposing said at least one type of crystal to a changing electromagnetic field is applied in a pulsed waveform;

98. The process according to claim 95, wherein at least one of the following holds true:
a. said process comprising an additional step of providing at least one type of crystal made of sucrose, silicon, pearl, smart pearl, quartz, smart patches, smart particles, encoded gems, encoded stones, encoded food supplements or any other inert material and a combination thereof;
b. said process comprising an additional step of providing an aqueous preparation in the form of a lotion, mixture, compound, formulation, gel, cream, liquid infusion, spray, suspension, emulsion, emollient preparation, emollient moisturiser and any combination thereof;
c. said process complies with GMP reproducibility standards; and
d. the final composition product meets the standard GMP requirements.

99. A method for prevention, treatment, stimulation and/or recovery of at least a portion of a mammalian tissue or system, preferably the skin system, wherein said method comprising the steps of:
a. providing a composition according to claim 1, and
b. applying said composition topically to at least a portion of the integuments, covering or support tissues of mammals so as to prevent, treat, and/or stimulate the recovery of said at least a portion of a mammalian tissue or system, preferably the skin system.

100. The method according to claim 99, wherein at least one of the following holds true:
a. comprising an additional step of applying said changing electromagnetic field according to a predetermined protocol;
b. comprising an additional step of exposing said at least one type of crystal to a changing electromagnetic field oscillating at a frequency in the range of between about 0.1 and 1000 Hz;
c. comprising an additional step of exposing said at least one type of crystal to a changing electromagnetic field oscillating at a frequency in the range of between about 0.2 and 200 Hz;
d. comprising an additional step of exposing said at least one type of crystal to a changing electromagnetic field oscillating at a frequency in the range of between about 1 and 150 Hz; and
e. comprising an additional step of applying said changing electromagnetic field in increased frequencies.

101. The method according to claim 99, wherein at least one of the following holds true:
a. comprising an additional step of applying said changing electromagnetic field in a series of predetermined intensities in the range of 1 and 1000 μT;
b. comprising an additional step of applying said changing electromagnetic field in a series of predetermined intensities in the range of 10 and 500 μT, preferably 5 to 200 μT;
c. comprising an additional step of applying said changing electromagnetic field in a continuous waveform;
d. comprising an additional step of applying said changing electromagnetic field in a pulsed waveform; and
e. comprising an additional step of providing said crystal made of sucrose, silicon, pearl, smart pearl, quartz, smart patches, smart particles, encoded gems, encoded stones, encoded food supplements or any other inert material and a combination thereof;

102. The method according to claim 99, wherein at least one of the following holds true:
a. comprising an additional step of preparing said aqueous preparation is in the form of a lotion, mixture, compound, formulation, gel, cream, liquid infusion, spray, suspension, emulsion, emollient preparation, emollient moisturiser and any combination thereof;
b. comprising an additional step of improving or treating at least one medical parameter or condition in a mammal, preferably in a human;
c. comprising an additional step of preventing at least one medical condition in a mammal, preferably in a human;
d. comprising an additional step of stimulating the regeneration and/or the recovery of an organ, tissue or a system or a portion thereof in a mammal, preferably in a human;
e. comprising an additional step of selecting said medical parameter or condition from the group consisting of skin related parameters and/or disorders, recovery of an organ, tissue or system or a portion thereof, brain related parameters and any combination thereof; and
f. comprising an additional step of applying said composition topically to the integuments and covering or support tissues of mammals, in human or veterinary fields.

103. The method according to claim 99, wherein at least one of the following holds true:
a. comprising an additional step of selecting said skin related parameters and/or disorders from the group consisting of trophism of the integumentary system, regeneration of skin cells, skin irritation, skin infection, skin inflammation, the effectiveness of the skin as a barrier, skin aging, skin pigmentation, allergy, dermatitis, allergic dermatitis, skin degeneration or atrophy and any combination thereof;
b. comprising an additional step of selecting said organ, tissue or system from the group consisting of the lymph system, the endocrine system, the liver, skin, the integumentary system and any portion or combination thereof; and
c. comprising an additional step of selecting said brain related parameters from the group consisting of regulation and/or controlling of the muscle tonus center and/or motor control center of the cerebellum, the reflex centers, regulation or controlling of the posterior pituitary (Neurohypophysis) of the pituitary gland or hypophysis, regulation of the skin center in the brain and any combination thereof.

104. An irradiated crystal activated by exposure to a changing electromagnetic field, for topical use.

105. The irradiated crystal according to claim 104, wherein said irradiated crystal is useful in cosmetic preparations.

106. The irradiated crystal according to claim 104, wherein at least one of the following holds true:
a. said irradiated crystal is useful in aqueous cosmetic preparations;
b. said changing electromagnetic field is applied according to a predetermined protocol;
c. said changing electromagnetic field oscillates at a frequency in the range of between about 0.1 and 1000 Hz;
d. said changing electromagnetic field oscillates at a frequency in the range of between about 0.2 and 200 Hz; and
e. said changing electromagnetic field oscillates at a frequency in the range of between about 1 and 150 Hz.

107. The irradiated crystal according to claim 104, wherein at least one of the following holds true:
a. said changing electromagnetic field is applied in increased frequencies;
b. said changing electromagnetic field is applied in a series of predetermined intensities in the range of 1 and 1000 μT; and
c. said changing electromagnetic field is applied in a series of predetermined intensities in the range of 10 and 500 μT, preferably 5 to 200 μT;

108. The irradiated crystal according to claim 106, wherein said changing electromagnetic field is applied in a continuous waveform.

109. The irradiated crystal according to claim 106, wherein said changing electromagnetic field is applied in a pulsed waveform.

110. The irradiated crystal according to claim 106, wherein said crystal is selected from the group consisting of sucrose, silicon, pearl, smart pearl, quartz, smart patches, smart particles, encoded gems, encoded stones, encoded food supplements or any other inert material and a combination thereof.

111. The irradiated crystal according to claim 106, wherein said aqueous preparation is in the form of a lotion, mixture, compound, formulation, gel, cream, liquid infusion, spray, suspension, emulsion, emollient preparation, emollient moisturiser and any combination thereof.

112. The irradiated crystal according to claim 106, wherein said aqueous preparation is applied topically to the integuments and covering or support tissues of mammals, in human or veterinary fields.
